(19) United States
(12) Patent Application Publication
(10) Pub. No.: US 2019/0076467 A1
(43) Pub. Date: Mar. 14, 2019

(54) ANTIVIRAL CONJUGATES OF POLYANIONIC POLYMER AND ANTIVIRAL DRUG

(71) Applicants: AARHUS UNIVERSITET, Aarhus C (DK); Commonwealth Scientific and Industrial Research Organisation, Acton (AU); REGION MIDTJYLLAND, Viborg (DK)

(72) Inventors: Alexander N. ZELIKIN, Aarhus N (DK); Martin TOLSTRUP, Risskov (DK); Kaja ZUWALA, Brabrand (DK); Benjamin M. WOHL, København Ø (DK); Anton A. A. SMITH, San Francisco, CA (US); Pau RUIZ-SANCHIS, Aarhus C (DK); Mille KRYGER, Odder (DK)

(73) Assignee: Commonwealth Scientific and Industrial Research Organisation, Acton (AU)

(21) Appl. No.: 15/580,898

(22) PCT Filed: Jun. 9, 2015
(86) PCT No.: PCT/DK2015/050154

§ 371 Date: Dec. 8, 2017

(51) Int. Cl. A61K 31/78; A61K 31/7056; A61K 47/58

(52) U.S. Cl. CPC A61K 31/78; A61K 47/58; A61K 31/7056

(57) ABSTRACT

A compound is provided, which comprise an anionic polymer carrier conjugated to an antiviral drug via a biodegradable linker. This compound is particularly useful as a broad-spectrum antiviral agent.

### REFERENCE TO EARLIER FILED APPLICATION

This application is a 371 national phase of PCT/DK2015/050154, filed June 9, 2015, the disclosure of which is incorporated, in its entirety, by this reference.

### FIELD OF INVENTION

The present invention relates to polymeric conjugates with antiviral activity.

### BACKGROUND OF INVENTION

Viral infections constitute a tremendous socio economic burden in agricultural, veterinary, and healthcare sectors. The costs of containment of the viral epidemics are enormous; pandemics may result in closure of borders of entire countries. One of the major drawbacks of the current antiviral tools is the lack of efficient antiviral agents, in particular broad spectrum agents. Development of curative agents and vaccines typically lags behind the advent and spread of viral infections and once developed, these agents are typically highly specific to the virus species and isotype such that e.g. for influenza, re-engineering of vaccines is required yearly. Furthermore, antibodies and biological agents are expensive to produce and further expensive to distribute using the cold chain of transportation.

Over the past few years, there has been some focus on the development of macromolecular (pro)drugs (MP) against hepatitis C and human immunodeficiency viruses (HCV and HIV, respectively). Other accomplishments in polymer research revealed that polymers are markedly cheaper than biological antiviral agents and can be synthesized on a large scale in a short period of time; do not typically require cold chain of transportation. For these reasons, a macromolecular (pro)drug shows promise asan antiviral treatment and its administration may help to alleviate the burden associated with viral diseases.

Polymers with anionic charge were shown to be effective against diverse viruses several decades ago [A. A. A. Smith, M. B. L. Kryger, B. M. Wohl, P. Ruiz-Sanchis, K. Zuwala, M. Tolstrup, A. N. Zelikin, Polym Chem-Uk 2014, 5, 6407]. It is accepted that polyanions are effective in preventing viral cell entry through electrostatic association with the viral particles, i.e. exert extracellular activity. [V. Pirrone, B. Wigdahl, F. C. Krebs, Antiviral Research 2011, 90, 168; M. Luscher-Mattli, Antivir Chem Chemoth 2000, 11, 249]. However clinical trials of polyanions as injectable antiviral agents have failed: sustained antiviral activity in blood requires high polymer concentrations in plasma maintained over long periods of time, and this commonly leads to increased toxicity.

### SUMMARY OF INVENTION

The present invention generally relates to compounds comprising or consisting of anionic polymers, in particularly polymerswhich comprise active drug agents coupled to the polymeric carrier via biodegradable linkers.

In a main aspect, a compound is provided which comprises a polyanion carrier conjugated to an antiviral drug via a biodegradable linker.

In another aspect, a compound comprising a polyanion carrier conjugated to an antiviral drug via a biodegradable linker is provided for use in medicine.

In yet another aspect a compound comprising a polyanion carrier conjugated to an antiviral drug via a biodegradable linker is provided for use as an antiviral agent.

These compounds are particularly useful as broad-spectrum antiviral agents because the compound has antiviral activity as a result of multiple modes of action, in particular the combination of any of the following antiviral activities:
i) extracellular inhibition of virus cell entry due to the activity of polyanion carrier;
ii) intracellular inhibition of the viral polymerases due to activity of the polyanion carrier; and
iii) intracellular antiviral activity due to release of said antiviral drug from the carrier upon cell entry.

Therefore, the compounds are useful in the treatment, amelioration or prevention of a retroviral infection or a Group V ((−)ssRNA viral infection. Specifically, the compound is provided for use in the treatment, amelioration or prevention of any of influenza, HIV, hepatitis C virus, ebola, mumps, respiratory syncytial virus, dengue and/or measles.

The biodegradable linker preferably comprises a disulfide bond and a self-immolative spacer, where the disulfide bond serves as a trigger for decomposition and drug release. Here, the linker is capable of releasing the antiviral drug via disulfide reshuffling in the presence of a thiol.

The antiviral drug is preferably a nucleoside or ribonucleoside analogue, for example, the drug can be ribavirin, azdothymidine, favipiravir and/or lamivudine or a derivative thereof.

The antiviral drug is preferably ribavirin.

In another embodiment, the antiviral drug is preferably favipiravir

The monomers of the compound are preferably ethylenically unsaturated monomers.

The monomers of the compound can be selected from the group consisting of poly(acrylic acid) or poly(methacrylic) acid.

The molar mass of the polyanion carrier is preferably between 3 and 30 kDa and the drug load is preferably between 1 and 40 mol %. A drug load of 40 mol % means that 40 out of 100 monomer comprise the drug.

In a further aspect, a method is provided of treating a viral infection comprising administering a compound comprising a polyanion carrier conjugated to an antiviral drug via a biodegradable linker to a subject in need thereof. This method can in a preferred embodiment be applied to human subjects in need of antiviral treatment, however, the same method may be applied to non-human animals in need of antiviral treatment, such as assinine (donkey), bovine (cattle), canine (dog), equine (horse), elaphine (deer), feline (cat), hircine (goat), leporine (rabbit,hare), murine (rodent), piscine (fish), or a porcine (pig) subject, preferably a porcine (pig), bovine (cattle), equine (horse) or hircine (goat) subjects.

The biodegradable linker preferably comprises of a disulfide bond and a self-immolative spacer, wherein the disulfide bond serves as a biodegradable trigger for intracellular decomposition and drug release via disulfide reshuffling in the presence of a thiol.

In yet another aspect, a method is provided for producing a compound comprising a polyanion carrier conjugated to antiviral drug via a biodegradable linker, said method comprising
a) synthesizing a conjugated anionic monomer containing an antiviral drug, wherein said monomer comprises a polymerizable double bond, and wherein said monomer is linked to the antiviral drug via biodegradable linker, and
b) polymerizing said conjugated anionic monomer with an anionic co-monomer.

In a preferred aspect said monomer comprises an ethylenically unsaturated monomer. The biodegradable linker preferably comprises both a disulfide bond and a self-immolative spacer and therefore, the method preferably comprises the steps of
a) synthesizing a conjugated anionic monomer containing an antiviral drug, wherein said monomer comprises a polymerizable double bond, and wherein said monomer is linked to the antiviral drug via a self-immolative spacer and a disulfide bond, and
b) polymerizing said conjugated anionic monomer with an anionic co-monomer.

Thus, in step a), the monomer and said antiviral drug is connected by a linker, which comprise a self-immolative spacer and a disulfide bond. It is preferred that the antiviral drug sits pendant to the polyanion carrier; i.e. that the drug is not part of the polymer backbone. In one preferred embodiment, the polyanion carrier is a linear polymer chain with pendant drugs attached to the backbone.

Most often, the anionic co-monomer corresponds to the anionic monomer of step a) without a coupled antiviral drug.

The preferred method for polymerization is a living polymerisation technique, most preferably reversible addition-fragmentation chain-transfer (RAFT).

### DESCRIPTION OF DRAWINGS

FIG. 1. Macromolecular characteristics of the polymers (number average molar mass (Mn) and content of ribavirin (RBV, mol %). C: viability of cells incubated with the polymers at the highest concentration tested (200 mg/L) over 24 h; (D-F) Infectivity of HIV in TZM-bl cells in the presence of macromolecular prodrugs at concentration 200 (D), 20 (E) and 2 (F) mg/L.

FIG. 2. Therapeutic activity and associated cell viability for (pro)drugs based on PAA and PMAA with regards to replication of the viral RNA for HCV replicon system hosted in Huh7 cells. Cells were incubated with the polymers (200 mg/L) for 48 h. Shown results are the mean ±SD of three independent experiments for PAA (n=3) and of two independent experiments for PMAA (n=2).

FIG. 3. Results of the quantitative polymerase chain reaction performed in the presence of negatively charged macromolecular (pro)drugs of ribavirin. PCR yields were normalized to the yield of PCR in the absence of added polymers. Results are shown as mean ±St. Dev. (N=3)

FIG. 4. Agarose gel electrophoresis analysis of the polymerase chain reaction performed using Taq polymerase in the presence of macromolecular (pro)drugs at concentration (200 mg/L) for polyanionic PAA (top line) and PMAA (bottom line) and non-ionic PVP (W, X) and HPMA (Y, Z). PTC—positive control, NTC—negative control.

FIG. 5. Results of the reverse transcriptase reaction performed in the presence of negatively charged macromolecular (pro)drugs of ribavirin. Reaction yields were normalized to the yield of positive control in the absence of added polymers. Results are shown as mean ±St. Dev. (N=3)

FIG. 6. Overview of RNA and DNA viruses

FIG. 7. Influenza

FIG. 8. Inhibition of vial infectivity in chicken embryos (Influenza PR8, Day 10 read out. Polymer (approx 2 mg/L based on allantoic fluid of 6 ml) and 500 pfu PR8 injected into Allantoic fluid same day; Incubated for 48 h; 5 embryos per group

FIG. 9. Respiratory syncytial virus (RSV) inhibition

FIG. 10. Measles

FIG. 11. Measles

FIG. 12. Mumps

FIG. 13. Ebola

### DETAILED DESCRIPTION OF THE INVENTION

### Macromolecular (Pro)Drug

The present disclosure relates to a macromolecular drug, comprising a polymeric carrier coupled to an antiviral agent. The antiviral activity of the drug can be attributed to the combined effects of the antiviral activity of the anionic carrier polymer and the antiviral activity of the conjugated antiviral drug.

The macromolecular drugs have different modes of action when used medically. In one aspect, the drug and the polymeric compound, as a whole, have antiviral properties. In another aspect, the compound acts as a prodrug, in that once administered the compound is subsequently converted from a pharmacologically inactive form to an active form through a normal metabolic process. The conversion occurs as a result of the presence of the self-immolative linkers, linkers which are commonly used in polymeric release technology. These types of linkers become labile upon activation, leading to the rapid disassembly of the drug from the parent polymer and thus the drug is released once the polymer has entered a cell. Thus, the term “self-immolative” is used synonymous with “self-degradable”. This release mechanism minimizes the potential toxicity of the antiviral drug. In the context of the present invention, the term macromolecular drug and macromolecular (pro)drug is intended to encompass both the direct drug effect and the prodrug effect of the compounds, unless specifically stated otherwise.

A main advantage of the macromolecular (pro)drug provided herein is that there is the ability to engineer into the same polymer chain at least three modes of antiviral activity, these being
i) extracellular inhibition of virus cell entry due to the activity of carrier anionic polymer;
ii) intracellular inhibition of any viral polymerases due to the activity of the carrier anionic polymer; and
iii) intracellular effect due to the antiviral drug released from the carrier upon cell entry.

Because of the multiple modes of antiviral actions, the drugs of the present invention are useful for the treatment of a wide range of viruses, and can also be used as broad spectrum antiviral agents.

### Polymer

The compounds provided herein are generally macromolecular drugs, which comprise a carrier polymer conjugated to an antiviral agent via a biodegradable linker. The polymer carrier is an anionic polymer; i.e. the carrier preferentially comprises monomers with negative charge. Such a polymer is also designated a polyanion herein.

The size of the polymer may vary depending on the specific use and route of administration. Generally, the size of the polyanion carrier may vary from 1 kDa up more than 1 MDa. In one range, the size of the polyanion carrier may vary from 1-500 kDa, such as 1-400 kDA, such as 1-300 kDA, such as 1-200 kDA, such as 1-100 kDA, such as 1-90 kDA, such as 1-80 kDA, such as 1-70 kDA, such as 1-60 kDA, such as 1-50 kDA, such as 1-40 kDA, such as 1-30 kDA,. In one such preferred embodiment, the molar mass of the polymer carrier is between 2 and 30 kDa, such as between 5 and 20 kDa. The molar mass of the polymer is for example 1, 2, 3, 4, 5, 6,7 ,8, 9, 10, 11, 12, 13, 14, 15, 16,17, 18, 19, 20, 21, 22, 23, 24 or 25 kDa. For example, the compound has a molar mass of about 7, 14 or 23 kDa. In a preferred embodiment, the polymer, such as a poly(acrylic) (PAA polymer), has a molecular weight in a range 7.3 to 9.5 kDa.

In general, larger polymers can be used for administration by inhalation, because inhaled polymers are not removed by kidney filtration. Thus, in general, larger polymers are preferred for administration by inhalation, which is the preferred route of administration for the treatment or use in treatment of respiratory viruses, in particular influenza and respiratory syncytial virus. Larger polymers are meant to include those above 10.000 kDa, for example those in the range of 10.000-100.000 kDa, such as 20.000-100.000 kDa, such as 30.000-100.000 kDa, such as 40.000-100.000 kDa, such as in the range of 50.000-100.000 kDa, such as 60.000-100.000 kDa, 70.000-100.000 kDa, such as 80.000-100.000 kDa, such as 90.000-100.000 kDa, or for example in the range of 10.000-50.000 kDa, such as 20.000-50.000 kDa, such as 30.000-50.000 kDa, such as 40.000-50.000 kDa, or in the range of 50.000-90.000 kDa, 50.000-80.000 kDa, such as 50.000-70.000 kDa, such as 50.000-60.000 kDa.

In contrast, smaller polymers are preferred for administration by injection, because smaller polymers are not cleared from circulation by the kidneys. Thus, for polymers, which are administered by injection, such as parenteral injection, for example intravenous injection, it is preferred that the molecular size is small enough to escape kidney clearance. Administration by injection is the preferred route of administration for the treatment or use in treatment of a number of viruses, including Hepatitis C, HIV and ebola. Smaller polymers are meant to include those below 30.000 kDa, such as those in the range of 1.000-30.000 kDa, such as 2.000-30.000 kDa, such as 3.000-30.000 kDa, such as 4.000 -30.000 kDa, such as 5.000-30.000 kDa, such as 6.000-30.000 kDa, such as 7.000-30.000 kDa, such as 8.000-30.000 kDa, such as 9.000-30.000 kDa, such as 10.000-10.000 kDa, such as 11.000-30.000 kDa, such as 12.000-30.000 kDa, such as 13.000-30.000 kDa, such as 14.000-30.000 kDa, such as 15.000-30.000 kDa, such as 20.000-30.000 kDa, such as 25.000-30.000 kDa.

In one specific embodiment, the polyanion carrier comprises monomers that are ethylenically unsaturated monomers, most preferably selected from selected from the group consisting of poly(acrylic acid) or poly(methacrylic acid). For example, the polyanion carrier is a poly(acrylic acid) polymer or a poly(methacrylic acid) polymer.

The polymer may comprise a mixture of different monomer structures, however in a preferred embodiment, the polyanion carrier comprise identical monomeric structures. However, individual monomers may vary in terms of drug loading, because the antiviral agent is not conjugated to all monomers of the carrier but to a smaller subset, such as 1-50 mol % or more, cf. below.

The polymers used herein may be linear, branched, hyperbranched, and/or dendritic polymers. However, in a preferred embodiment, the polymer is a linear polymer.

In one preferred embodiment, the provided compound comprise a PMAA polyanion carrier conjugated to an antiviral drug, such as ribavirin, via a biodegradable linker, wherein the molecular weight of the compound is between 10-20 kDa, such as between 12-18, such as preferably 13-15, such as most preferred 14 kDa and the drug load is 2-10 mol %, such as preferably around 4 mol %. In another embodiment, the provided compound comprise a PMAA polyanion carrier conjugated to an antiviral drug, such as ribavirin, via a biodegradable linker, wherein the molecular weight of the compound is between 20-35 kDa, such as between 25-35, such as preferably 25-30, such as most preferred 28 kDa and the drug load is 2-10 mol %, such as preferably around 5 mol %.

### Linker

A key feature of the polymeric compounds of the invention is the use of a reversible linker. The polyanion and antiviral drug is conjugated via a biodegradable linker that contains a labile, self-immolative spacer that allows controlled release of the antiviral drug. Thus, the biodegradable linker is capable of releasing the conjugated antiviral drug in a controlled manner. A number of such linkers are available. For example biodegradablelinkers comprising ester linkages can be used, where a conjugated drug agent can be released by hydrolysis of the ester. The linker also comprises an entity, which triggers the decomposition of the self immolative linker and drug release. Such a trigger is for example a disulfide bond. Thus, in one preferred embodiment, the linker comprises a disulfide bond. A linker comprising a disulfide bond is capable of releasing the conjugated antiviral agent via disulfide reshuffling in the presence of a thiol. Thus, the disulfide bond can work as a trigger that initiates the decomposition of the self immolative linker. In this manner, a polyanion conjugate, which incorporates an antiviral drug, undergoes self-immolative cleavage when exposed to biological thiols. This leads to the intracellular release of the antiviral drug. Thus, in a preferred embodiment, the biodegradable linker comprises a self-immolative linker and a disulfide bond.

One preferred biodegradable linker has the following structure:

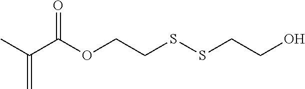

This biodegradable linker can be conjugated to an antiviral drug. For example, the linker may be conjugated to ribavirin to form the following intermediate compound:

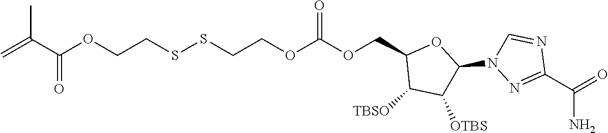 [SMILES: C=C(C)C(=O)OCCSSCCOC(=O)OC[C@H]1O[C@@H](n2cnc(C(N)=O)n2)[C@H](O[Si](C)(C)C(C)(C)C)[C@@H]1O[Si](C)(C)C(C)(C)C]

The prodrugs built around this linkage are stable in the blood stream and degrade upon cell entry in response to the intracellular concentration of glutathione, GSH.

### Antiviral Drug Agent

The antiviral polymeric compounds provided herein are characterized in that an antiviral drug is conjugated to a polyanionic carrier via a self-immolative linker. This provides an additional mode of antiviral action of the polymer. The polyanion per se provides for extracellular inhibition of virus cell entry and intracellular inhibitsviral polymerases. Additionally, the conjugated drug agent provides the polymeric compound with an additional mode of action, namely an intracellular antiviral activity due to the direct activity of anthe tiviral drug, which is released from the carrier upon cell entry.

The controlled release of the antiviral drug also minimizes the potential toxicity inherent in the free drug, as it is only released from polymer at the required treatment site and only uponactivation.

The antiviral drug is preferably a drug with broad-spectrum antiviral activity. The antiviral drug is in one embodiment, a nucleoside or nucleoside analogue or ribonucleoside analogue. Examples of such analogues include:
deoxyadenosine analogues, such as Didanosine (ddl)(HIV)
adenosine analogues, such as BCX4430 (Ebola)
deoxycytidine analogues, such as Emtricitabine (FTC)(HIV), Lamivudine (3TC)(HIV, hepatitis B) and Zalcitabine (ddC)(HIV)
guanosine and deoxyguanosine analogues, such as Abacavir (HIV), Aciclovir and Entecavir (hepatitis B)
thymidine and deoxythymidine analogues, such as Stavudine (d4T), Telbivudine (hepatitis B) and Zidovudine (azidothymidine, or AZT)(HIV)
deoxyuridine analogues, such as Idoxuridine and Trifluridine

Thus, in one embodiment, the conjugated antiviral drug is selected from the group consisting of analogue of deoxyadenosine, adenosine, Deoxycytidine, guanosine, Thymidine and deoxyuridine, as specified above.

In one specific embodiment, the antiviral drug is selected from the group consisting of ribavirin, azidothymidine and lamivudine.

In a preferred embodiment of the compound provided herein, the polyanion is conjugated to ribavirin or a derivative thereof.

Ribavirin:

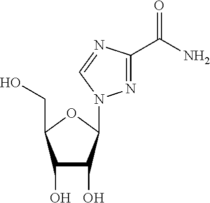

Ribavirin is broad-spectrum antiviral drug. It exhibits antiviral activity against a broad range of RNA viruses and is used clinically for the treatment to hepatitis C virus infections, respiratory syncytial virus infections, viral hemorrhagic fevers and Lassa fever virus infections, but also for other viral infections. It is a guanosine (ribonucleic) analogue used to stop viral RNA synthesis and viral mRNA capping, thus, it is a nucleoside inhibitor. Its brand names include Copegus, Rebetol, Ribasphere, Vilona, and Virazole.

Ribavirin is also a prodrug, which resembles purine RNA nucleotides after being metabolized. In this metabolized form it interferes with RNA metabolism and induces mutations during DNA replication.

Ribavirin appears on the World Health Organization list of essential medicines for both adults and children. Although, the drug is effective against a number of viruses it has proved to be insufficient as monotherapy against Hepatitis C virus (HPC) or HIV. Furthermore, hematotoxicity of ribavirin makes the drug markedly less attractive for widespread use. However, conjugation to a polymer prevents the entry of ribavirin into red blood cells thus eliminating the main cause of the side effects of the drug.

In another specific embodiment, the conjugated antiviral drug is favipiravir or a functional derivative thereof. Favipiravir is also known as T-705 or Avigan. The chemical structure of favipiravir is shown below:

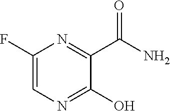 [SMILES: NC(=O)c1nc(F)cnc1O]

The antiviral drug has been developed by Toyama Chemical of Japan and has proven active against many RNA viruses. It is active against influenza viruses, West Nile virus, yellow fever virus, foot-and-mouth disease virus as well as other flaviviruses, arenaviruses, bunyaviruses and alphaviruses. Activity against enteroviruses and Rift Valley fever virus has also been demonstrated. Favipiravir is a pyrazinecarboxamide derivative.

Favipiravir appears to selectively inhibit viral RNA-dependent RNA polymerase. Importantly, favipiravir does not inhibit RNA or DNA synthesis in mammalian cells and is not toxic to them.

The polymer carrier consists of a chain of interlinked monomers. This chain comprises both normal (pristine) monomers (without a linked antiviral drug) and monomers conjugated to an antiviral drug such as ribavirin via a biodegradable linker. The drug load on the polymer carrier may affect the activity of the compounds presented herein.

The drug load may reach 50 mol % or more. Thus, the drug load may vary between 1-50 mol %, such as between 1-40 mol %, such as between 5-40 mol %, such as between 10-40 mol %. However, normally, the drug load varies between 1-30 mol %, such as between 1-20 mol %, such as between 5-15 mol %.

### Process for Preparation of Polymer Conjugate

In one specific aspect, a process is provided for producing a macromolecular (pro)drug of the present invention. Generally, a method is provided for producing a compound consisting of a polyanion carrier conjugated to antiviral drug via a self-immolative linker. This method comprises the steps of
a) synthesizing an anionic monomer containing an antiviral drug, wherein said monomer comprises a polymerizable double bond, which is linked to the antiviral drug via a self-immolative linker, and
b) polymerizing the above monomer with an anionic co-monomer.

The self-immolative linker preferably comprises a disulfide bond.

The co-monomer, may be selected from any suitable anionic monomer, however, in general it is often convenient to polymerize the conjugated monomer with an anionic co-monomer corresponding to the conjugated monomer of step a); i.e. an identical monomer, but without a coupled antiviral drug. Thus, a polymethacrylate (PMAA) monomer conjugated to an antiviral drug, such as ribavirin is preferably polymerized with PMMA as co-monomer; and a poly(acrylic acid) (PAA) monomer conjugated to an antiviral drug, such as ribavirin, is preferably polymerized with PAA as co-monomer.

Any available method may be used for polymerization. For example, any living polymerization technique may be employed as well as any controlled radical polymerization technique. In one embodiment, polymerization is performed by a reversible addition-fragmentation chain-transfer (RAFT), atom transfer radical polymerization (ATRP) or nitroxide-mediated polymerization (NMP).

In one preferred embodiment, polymerization is performed by reversible addition-fragmentation chain-transfer (RAFT).

RAFT polymerization is one of several kinds of Reversible-deactivation radical polymerization. It makes use of a chain transfer agent in the form of a thiocarbonylthio compound (or similar) to afford control over the generated molecular weight and polydispersity during a free-radical polymerization of one or more more ethylenically unsaturated monomers so as to form a living polymer chain (i.e. a polymer chain that has been formed according to a living polymerisation techniqueThus, RAFT polymerization can use thiocarbonylthio compounds, such as dithioesters, thiocarbamates, and xanthates, to mediate the polymerization via a reversible chain-transfer process. Examples of RAFT agents are sold by Boron Molecular, Sigma Aldrich, Strem to name a few and are also described in WO201083569 and Benaglia et al, Macromolecules. (42), 9384-9386, 2009, the entire contents of which are incorporated herein by reference.). As with other controlled radical polymerization techniques, RAFT polymerizations can be performed with conditions to favour low dispersity (molecular weight distribution) and a pre-chosen molecular weight.

Living polymerisation also allow for the production of polymer chains that comprise of block copolymers (blocks made up of different monomers), branched or comb polymers, stars or microgels, or simple linear backbone chains.

Examples of ethylenically unsaturated monomers include maleic anhydride, N-alkylmaleimide, K-arylmaleimide, dialkyl fumarate and cyclopolymerisable monomers, acrylate and methacrylate esters, acrylic and methacrylic acid, styrene, styrenics, methacrylamide, and methacrylonitnle, methyl methacrylate, ethyl methacrylate, propyl methacrylate (all isomers), butyl methacrylate (all isomers), 2-ethylhexyl methacrylate, isobornyl methacrylate, methacrylic acid, benzyl methacrylate, phenyl methacrylate, oligo (ethylene glycol) methyl ether methacrylate, methacrylonitnle, alpha-methylstyrene, methyl acrylate, ethyl acrylate, propyl acrylate (all isomers), butyl acrylate (all isomers), 2-ethylhexyl acrylate, isobornyl acrylate, acrylic acid, benzyl acrylate, phenyl acrylate, acrylonitrile, styrene, functional methacrylates, acrylates and styrenes selected from glycidyl methacrylate, 2-hydroxyethyl methacrylate, hydroxypropyl methacrylate (all isomers), hydroxybutyl methacrylate (all isomers), N,N-dimethylaminoethyl methacrylate, N,N-diethylaminoethyl methacrylate, triethyleneglycol methacrylate, itaconic anhydride, itaconic acid, glycidyl acrylate, 2-hydroxyethyl acrylate, hydroxypropyl acrylate (all isomers), hydroxybutyl acrylate (all isomers), N,N-dimethylaminoethyl acrylate, N,N-diethylaminOethyl acrylate, triethyleneglycol acrylate, methacrylamide, N-methylacrylamide, N,N-dimethylacrylamide, N-tert-butylmethacrylamide, N-n-butylmethacrylamide, N-methylolmethacrylamide, N-ethylolmethacrylamide, N-tert-butylacrylamide, N-n-butylacrylamide, N-methylolacrylamide, N-ethylolacrylamide, vinyl benzoic acid (all isomers), diethylamino styrene (all isomers), alpha-methylvinyl benzoic acid (all isomers), diethylamino alpha-methylstyrene (all isomers), p-vinylbenzene sulfonic acid, p-vinylbenzene sulfonic sodium salt, trimethoxysilylpropyl methacrylate, triethoxysilylpropyl methacrylate, tributoxysilylpropyl methacrylate, dimethoxymethylsilylpropyl methacrylate, diethoxymethylsilylpropyl methacrylate, dibutoxymethylsilylpropyl methacrylate, diisopropoxymethylsilylpropyl methacrylate, dimethoxysilylpropyl methacrylate, diethoxysilylpropyl methacrylate, dibutoxysilylpropyl methacrylate, diisopropoxysilylpropyl methacrylate, trimethoxysilylpropyl acrylate, triethoxysilylpropyl acrylate, tributoxysilylpropylacrylate, dimethoxymethylsilylpropyl acrylate, diethoxymethylsilylpropyl acrylate, dibutoxymethylsilylpropyl acrylate, diisopropoxymethylsilylpropyl acrylate, dimethoxysilylpropyl acrylate, diethoxysilylpropyl acrylate, dibutoxysilylpropyl acrylate, diisopropoxysilylpropyl acrylate, vinyl acetate, vinyl butyrate, vinyl benzoate, vinyl chloride, vinyl fluoride, vinyl bromide, N-phenylmaleimide, N-butylmaleimide, N-vinylpyrrolidone, N-vinylcarbazole, butadiene, ethylene and chloroprene. This list is not exhaustive.

Use as Antiviral Agents

The compounds comprising a polyanion carrier conjugated to an antiviral drug via a biodegradable linker are particularly useful in medicine and in therapeutic methods. In one aspect, the compound is provided for use in medicine. More specifically, the compound comprising a polyanion carrier conjugated to an antiviral drug via a biodegradable linker is provided for use as an antiviral agent. Thus, the compound according to any of the preceding claims for use in the treatment, amelioration or prevention of a retroviral infection.

A method is also provided of treating a viral infection comprising administering a compound comprising a polyanion carrier conjugated to an antiviral drug via a biodegradable linker to a subject in need thereof. Preferably, the subject is a human being in need of antiviral treatment. However, the subject may also be a non-human animal in need of antiviral treatment. For example the non-human animal can be selected from the group consisting of is an assinine (donkey), bovine (cattle), canine (dog), equine (horse), elaphine (deer), feline (cat), hircine (goat), leporine (rabbit,hare), murine (rodent), piscine (fish), or a porcine (pig) subject, preferably a porcine (pig), bovine (cattle), equine (horse) and a hircine (goat) subject.

The use of the compounds as antiviral agents is particularly advantageous because the compounds has antiviral activity by at least one, such as, two or preferably three modes of antiviral activities selected from i) extracellular inhibition of virus cell entry due to the activity of polyanion carrier; ii) intracellular inhibition of the viral polymerases due to activity of the polyanion carrier; and iii) intracellular antiviral activity due to release of said antiviral drug from the carrier upon cell entry. This means that the compounds provided herein are superior to other polymeric compounds, which only function through one or two of these mechanisms.

In one aspect, the compounds comprising a polyanion carrier conjugated to an antiviral drug via a biodegradable linker are provided for use in the manufacture of a medicament. In particular, these compounds are provided for use in the manufacture of a medicament for the treatment, amelioration and/or prevention of viral disorder.

In a preferred embodiment of the methods and uses provided herein, the biodegradable linker comprises both a disulfide bond and a self-immolative spacer. This biodegradable linker has a disulfide bond as a trigger for decomposition. This means that the linker is capable of releasing the conjugated antiviral drug via disulfide reshuffling in the presence of a thiol.

The compounds may be used for treatment, amelioration and/or prevention of any viral disorder. In one particular embodiment, the compounds are provided for use in the treatment, amelioration or prevention of a Group V ((−)ssRNA viral infection. Specifically, the compounds are provided for use in the treatment, amelioration or prevention of influenza, HIV, hepatitis C virus, ebola, mumps, respiratory syncytial virus, dengue and/or measles. Thus, the viral disorder may in one embodiment be selected from the group consisting of influenza, HIV, hepatitis C virus, ebola, mumps, respiratory syncytial virus, dengue and/or measles. In one preferred embodiment, the compounds are used in the treatment, amelioration or prevention of ebola. In another preferred embodiment, the compounds are used in the treatment, amelioration or prevention of hepatitis C virus. In yet another preferred embodiment, the compounds are used in the treatment, amelioration or prevention of HIV.

In one preferred embodiment, the provided compound for the treatment of influenza comprise a PMAA polyanion carrier conjugated to an antiviral drug, such as ribavirin, via a biodegradable linker, wherein the molecular weight of the compound is between 10-20 kDa, such as between 12-18, such as preferably 13-15, such as most preferred 14 kDa and the drug load is 2-10 mol %, such as preferably around 4 mol %. In another embodiment, the provided compound for the treatment of influenza comprise a PMAA polyanion carrier conjugated to an antiviral drug, such as ribavirin, via a biodegradable linker, wherein the molecular weight of the compound is between 20-35 kDa, such as between 25-35, such as preferably 25-30, such as most preferred 28 kDa and the drug load is 2-10 mol %, such as preferably around 5 mol %.

In one preferred embodiment, the provided compound for the treatment of hepatitis comprise a PMAA polyanion carrier conjugated to an antiviral drug, such as ribavirin, via a biodegradable linker, wherein the molecular weight of the compound is between 10-20 kDa, such as between 12-18, such as preferably 13-15, such as most preferred 14 kDa and the drug load is 2-10 mol %, such as preferably around 4 mol %. In another embodiment, the provided compound for the treatment of hepatitis comprise a PMAA polyanion carrier conjugated to an antiviral drug, such as ribavirin, via a biodegradable linker, wherein the molecular weight of the compound is between 20-35 kDa, such as between 25-35, such as preferably 25-30, such as most preferred 28 kDa and the drug load is 2-10 mol %, such as preferably around 5 mol %.

In one preferred embodiment, the provided compound for the treatment of ebola comprise a PMAA polyanion carrier conjugated to an antiviral drug, such as ribavirin, via a biodegradable linker, wherein the molecular weight of the compound is between 10-20 kDa, such as between 12-18, such as preferably 13-15, such as most preferred 14 kDa and the drug load is 2-10 mol %, such as preferably around 4 mol %. In another embodiment, the provided compound for the treatment of ebola comprise a PMAA polyanion carrier conjugated to an antiviral drug, such as ribavirin, via a biodegradable linker, wherein the molecular weight of the compound is between 20-35 kDa, such as between 25-35, such as preferably 25-30, such as most preferred 28 kDa and the drug load is 2-10 mol %, such as preferably around 5 mol %.

In one preferred embodiment, the provided compound for the treatment of respiratory syncytial virus comprise a PMAA polyanion carrier conjugated to an antiviral drug, such as ribavirin, via a biodegradable linker, wherein the molecular weight of the compound is between 10-20 kDa, such as between 12-18, such as preferably 13-15, such as most preferred 14 kDa and the drug load is 2-10 mol %, such as preferably around 4 mol %. In another embodiment, the provided compound for the treatment of respiratory syncytial virus comprise a PMAA polyanion carrier conjugated to an antiviral drug, such as ribavirin, via a biodegradable linker, wherein the molecular weight of the compound is between 20-35 kDa, such as between 25-35, such as preferably 25-30, such as most preferred 28 kDa and the drug load is 2-10 mol %, such as preferably around 5 mol %.

In one preferred embodiment, the provided compound for the treatment of measles comprise a PMAA polyanion carrier conjugated to an antiviral drug, such as ribavirin, via a biodegradable linker, wherein the molecular weight of the compound is between 10-20 kDa, such as between 12-18, such as preferably 13-15, such as most preferred 14 kDa and the drug load is 2-10 mol %, such as preferably around 4 mol %. In another embodiment, the provided compound for the treatment of measles comprise a PMAA polyanion carrier conjugated to an antiviral drug, such as ribavirin, via a biodegradable linker, wherein the molecular weight of the compound is between 20-35 kDa, such as between 25-35, such as preferably 25-30, such as most preferred 28 kDa and the drug load is 2-10 mol %, such as preferably around 5 mol %.

In one preferred embodiment, the provided compound for the treatment of mumps comprise a PMAA polyanion carrier conjugated to an antiviral drug, such as ribavirin, via a biodegradable linker, wherein the molecular weight of the compound is between 10-20 kDa, such as between 12-18, such as preferably 13-15, such as most preferred 14 kDa and the drug load is 2-10 mol %, such as preferably around 4 mol %. In another embodiment, the provided compound for the treatment of mumps comprise a PMAA polyanion carrier conjugated to an antiviral drug, such as ribavirin, via a biodegradable linker, wherein the molecular weight of the compound is between 20-35 kDa, such as between 25-35, such as preferably 25-30, such as most preferred 28 kDa and the drug load is 2-10 mol %, such as preferably around 5 mol %.

In one preferred embodiment, the provided compound for the treatment of dengue comprise a PMAA polyanion carrier conjugated to an antiviral drug, such as ribavirin, via a biodegradable linker, wherein the molecular weight of the compound is between 10-20 kDa, such as between 12-18, such as preferably 13-15, such as most preferred 14 kDa and the drug load is 2-10 mol %, such as preferably around 4 mol %. In another embodiment, the provided compound for the treatment of dengue comprise a PMAA polyanion carrier conjugated to an antiviral drug, such as ribavirin, via a biodegradable linker, wherein the molecular weight of the compound is between 20-35 kDa, such as between 25-35, such as preferably 25-30, such as most preferred 28 kDa and the drug load is 2-10 mol %, such as preferably around 5 mol %.

The treatment can be applied at any stage of viral infection and can also be used for prophylactic treatment of viral infections. Thus, the treatment according to the present invention involves both prophylactic treatment as well as curative treatment as well as prevention and amelioration of symptoms associated with a viral infection, as defined herein above.

The uses, medicaments and methods of treatment, amelioration and prevention provided herein could also be combined with other antiviral agents or treatments. Such agents are known to those of skill in the art. Potential agents include nucleoside and ribonucleoside analogues. For example, the use, medicament and/or method is combined with an additional antiviral agent selected from the group consisting of Abacavir, Aciclovir, Acyclovir, Adefovir, Amantadine, Amprenavir, Ampligen, Arbidol, Atazanavir, Atripla (fixed dose drug), Balavir, Cidofovir, Combivir (fixed dose drug), Dolutegravir, Darunavir, Delavirdine, Didanosine, Docosanol, Edoxudine, Efavirenz, Emtricitabine, Enfuvirtide, Entecavir, Ecoliever, Famciclovir, Fixed dose combination (antiretroviral), Fomivirsen, Fosamprenavir, Foscarnet, Fosfonet, Fusion inhibitor, Ganciclovir, Ibacitabine, Imunovir, Idoxuridine, Imiquimod, Indinavir, Inosine, Integrase inhibitor, Interferon type III, Interferon type II, Interferon type I, Interferon, Lamivudine, Lopinavir, Loviride, Maraviroc, Moroxydine, Methisazone, Nelfinavir, Nevirapine, Nexavir, Nucleoside analogues, Novir, Oseltamivir (Tamiflu), Peginterferon alfa-2a, Penciclovir, Peramivir, Pleconaril, Podophyllotoxin, Protease inhibitor (pharmacology), Raltegravir, Reverse transcriptase inhibitor, Ribavirin, Rimantadine, Ritonavir, Pyramidine, Saquinavir, Sofosbuvir, Stavudine, Synergistic enhancer (antiretroviral), Tea tree oil, Telaprevir, Tenofovir, Tenofovir disoproxil, Tipranavir, Trifluridine, Trizivir, Tromantadine, Truvada, Valaciclovir (Valtrex), Valganciclovir, Vicriviroc, Vidarabine, Viramidine, Zalcitabine, Zanamivir (Relenza), Zidovudine and favipiravir. In a preferred embodiment, the combined antiviral agent is azdothymidine, lamivudine and/or a derivative thereof.

### Administration

In general, suitable methods of administering the polymeric compounds provided herein are well-known in the art. Thus, any suitable route of administration may be employed for providing a mammal, especially a human, with an effective dose of a polyanion carrier conjugated to an antiviral drug as provided herein. For example, oral, rectal, vaginal, topical, parenteral, ocular, pulmonary, nasal, and the like may be employed. Other examples of administration include sublingual, intravenous, intramuscular, intrathecal, subcutaneous, cutaneous and transdermal administration. In one embodiment the administration comprises inhalation, injection or implantation. The administration of the polymeric compound can result in a local (topical) effect or a body-wide (systemic) effect. In a preferred embodiment, the polymeric compound is administered by orally.

Dosage forms include tablets, troches, dispersions, suspensions, solutions, capsules, creams, ointments, aerosols, and the like. The effective dose employed of a polymeric compound provided herein may vary depending on the particular compound, the mode of administration, the condition being treated and the severity of the condition being treated. Such dosage may be ascertained readily by a person skilled in the art.

In one embodiment the conjugated polymeric compound is administered at a dosage of from about 0.1 milligram to about 100 milligram per kilogram of body weight. For most large mammals, the total dosage is from about 1.0 milligrams to about 1000 milligrams, preferably from about 1 milligram to about 50 milligrams. In the case of a 70 kg adult human, the total dose will generally be from about 1 milligram to about 350 milligrams. For a particularly potent compound, the dosage for an adult human may be as low as 0.1 mg. The dosage regimen may be adjusted within this range or even outside of this range to provide the optimal therapeutic response.

The conjugated compound may be used for prophylactic treatment, and thus be administered to subjects, who are exposed to viral infection and therefore are at risk of contracting a viral infection. For example, the compound may be used as a gel applied to condoms for topical administration of the compound for prevention of sexually transmitted viruses, such as HIV and/or Hepatitis C.

However, the conjugated compounds are also effective in treating subjects who have contracted a viral disease. The symptoms of viral disease, such as influenza, HIV, hepatitis C virus, Ebola, mumps, respiratory syncytial virus, dengue and measles are well-known. Moreover, viral infections can be determined by immunology, i.e. in antibody-based methods. Thus, in one embodiment, the methods, compounds and uses thereof are provided for use in the treatment and/or amelioration of a viral infection, wherein the subject receiving the treatment has been tested positive for said viral infection, such as a viral infection selected from influenza, HIV, hepatitis C virus, Ebola, mumps, respiratory syncytial virus, dengue and measles.

### EXAMPLES

### Example 1

Macromolecular (pro)drugs fight HCV and HIV through a combination of three mechanisms

It has been previously observed that polyanions effectively suppress intracellular replication of a hepatitis C viral replicon. This approach however lacks an extracellular part of the virus life cycle and polymer activity is therefore limited to intracellular effects. Based on this observation, herein was developed antiviral macromolecular (pro)drugs (MP) with activity exerted by at least three independent mechanisms each of which is based on non-specific, universal antiviral modes of activity. The following examplesinvestigated the activity of these formulations against HCV and HIV.

The cornerstone in the design of these macromolecular (pro)drugs resulted form success in developing a process for the synthesis of libraries of macromolecular (pro)drugs with independent variations of the linear polymer backbone (acrylic acid, methacrylic acid, N-2-hydroxypropyl methacrylamide), molar mass, and content of conjugated an antiviral drug, ribavirin (RBV). Ribavirin is a broad-spectrum antiviral agent and is effective against a number of viruses, although insufficient as monotherapy against HCV or HIV. Furthermore, ribavirin (unconjugated) is hematotoxic and therefore unattractive for widespread, commercial use. The polymeric drugs provided herein re-establish ribavirin as an attractive drug because its conjugation to a polymer prevents its entry into red blood cells thus eliminating the cause of the main side effect of this drug. The synthesized macromolecular (pro)drugs were effective in counteracting inflammation, inhibiting replication of the viral genome within a HCV replicon, and suppressing infectivity of HIV—through activity of the released drug.

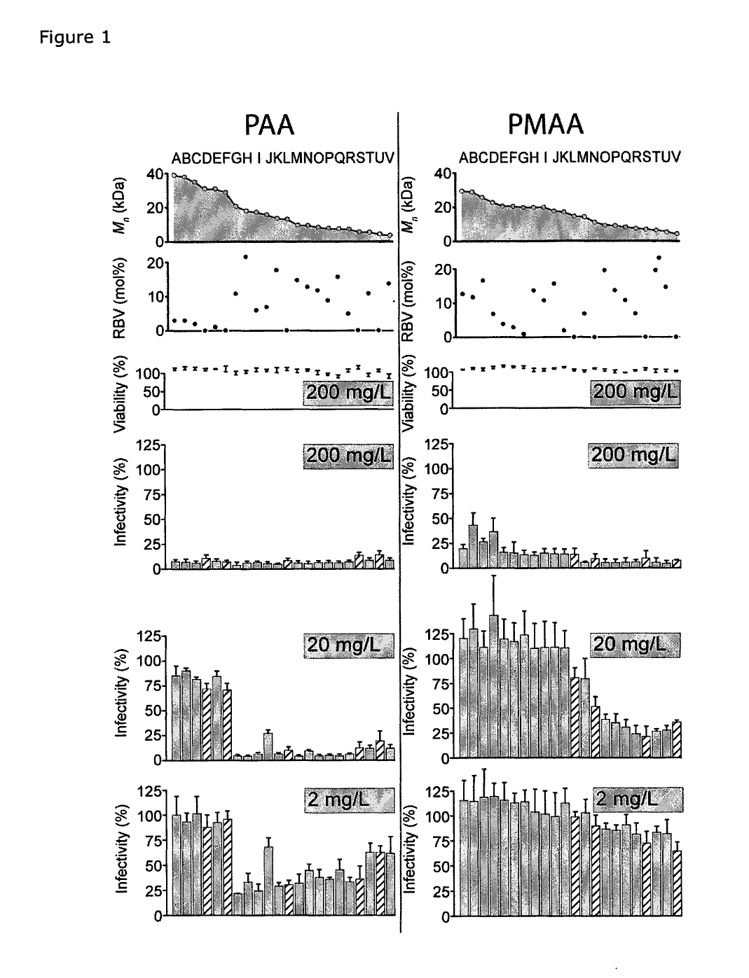

Further investigation of the mechanism of action of the polymeric drugs of ribavirin based on polyanions was then undertaken. The polymer libraries tested herein consisted of a 21 polymers based on poly(acrylic acid) and 21 counterpart based on poly(methacrylic acid) (PAA and PMAA, respectively). Polymers were synthesized via controlled radical polymerization to obtain samples with independently controlled molar mass and content of ribavirin (meth)acrylate. Infectivity of HIV was tested at polymer concentrations 2, 20, and 200 mg/L using a TZM-bl cell line, Bal-1 strain of HIV, and luciferase read-out, FIG. 1. At high polymer concentration, PAA and PMAA based (pro)drugs inhibited infectivity of HIV to a level below 10%. Decrease in polymer content resulted in an expected drop in activity of formulations and at 2 mg/L, PMA-based (pro)drugs lost vertically all of their efficacy in preventing infectivity of HIV. For PAA-family of polymers, drop in activity was less pronounced and even at 2 mg/L concentration, certain polymer compositions were highly effective and suppressed HIV infectivity to ˜20%. Higher apparent activity of acrylic polymers over their methacrylic counterparts could be readily explained by a higher anionic character of the former—pKa 4.5 vs 6.5 for acrylic and methacrylic acid, respectively. The ccrylate library explicitly reveals that both the polymer and the carrier contribute to the antiviral effect and e.g. at 2 mg/L, 20 kDa (pro)drug has a pronounced effect on HIV infectivity whereas the parent pristine polymer is devoid of such activity.

Interestingly, PAA and PMAA based libraries alike, the highest molar mass polymers were not the most effective, as would be expected for a mechanism of antiviral activity based solely on prevention of viral cell entry. Higher molar mass polymers exhibit higher affinity within their interpolyelectrolyte complexes and polyelectrolyte multilayers and would be expected to have higher affinity to the HIV viral particles. Enhanced activity of low molar mass polymers can be attributed to greater extent of polymer entry into cells. This observation would therefore suggest that against HIV polymers act through three mechanisms, prevention of viral cell entry (extracellular mechanism), intracellular activity of the released ribavirin, and intracellular activity of the polyanions inherent with their structure (anionic charge).

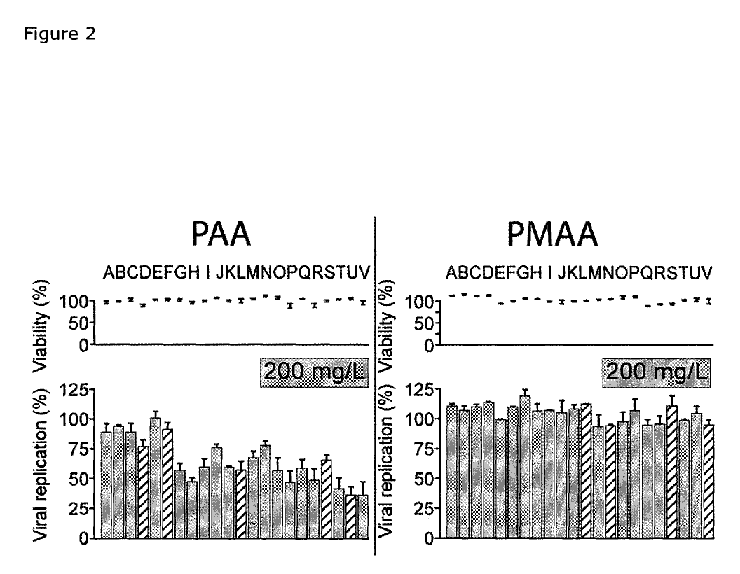

Next, the two libraries of polyanionic (pro)drugs of ribavirin were tested for activity against replication of the viral genome of HCV using a sub-genomic replicon of this virus. Replication of the viral genome is engineered to produce a copy of luciferase, the latter being readily quantified via measurements of luminescence. The replicon system does not produce replication competent viral particles and therefore can be handled in standard cell culture facilities. The above features make HCV replicon highly useful for the design and development of drugs against HCV. For the polyanionic (pro)drugs of ribavirin, we observed that PMAA-based prodrugs showed no activity against replication of the viral genome, FIG. 2. In stark contrast, PAA based counterparts revealed a high level of activity and suppressed replication of HCV genome to below 40%. This effect was clearly molar mass dependent. Highest molar mass polymers exhibited negligible antiviral activity, whereas shortest polymer chains revealed high efficiency of anti-HCV activity. We have previously observed the same trend of the PAA based library as used herein when evaluating delivery of ribavirin to macrophages and using anti-inflammatory activity of ribavirin as a read-out. {Smith, 2014 #4} Using flow cytometry, we showed that shorter chains exhibit a higher level of cell entry compared to the higher molar mass analogues with the same content of RIBAVIRIN. Similar considerations are likely to explain enhanced activity of PAA (pro)drug with lowest molar mass observed in FIG. 2.

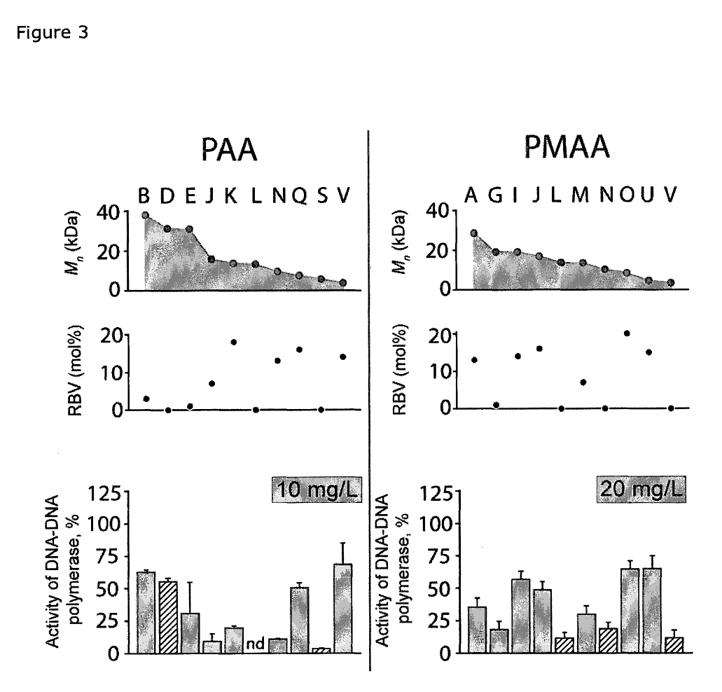

With regards to the mechanism of anti-HCV activity, experimental data suggest that pristine PAA and ribavirin-containing counterparts with similar molar mass have similar levels activity suggesting that in this case, it is the polyanionic nature of the carrier which contributes the most to the overall therapeutic effect. Replicon system as used herein lacks an extracellular part of the viral life cycle and only intracellular modes of activity are possible. Based on this, it appears that PAA/PMAA-based polyanionic (pro)drugs of ribavirin could draw exert activity through interference with activity of the viral polymerase. Polyanions may effectively compete with the viral nucleic acid for binding with the viral polymerase—if such binding has a strong electrostatic component. To provide direct evidence for activity of PAA/PMAA based (pro)drugs against polymerases, select polymers from these libraries were added to the standard mix of reagents for quantitative PCR reaction, FIG. 3.

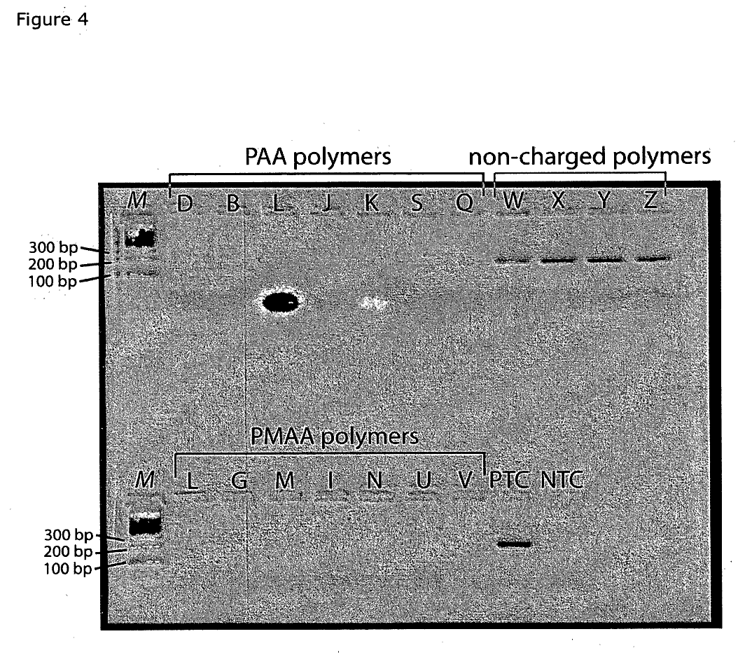

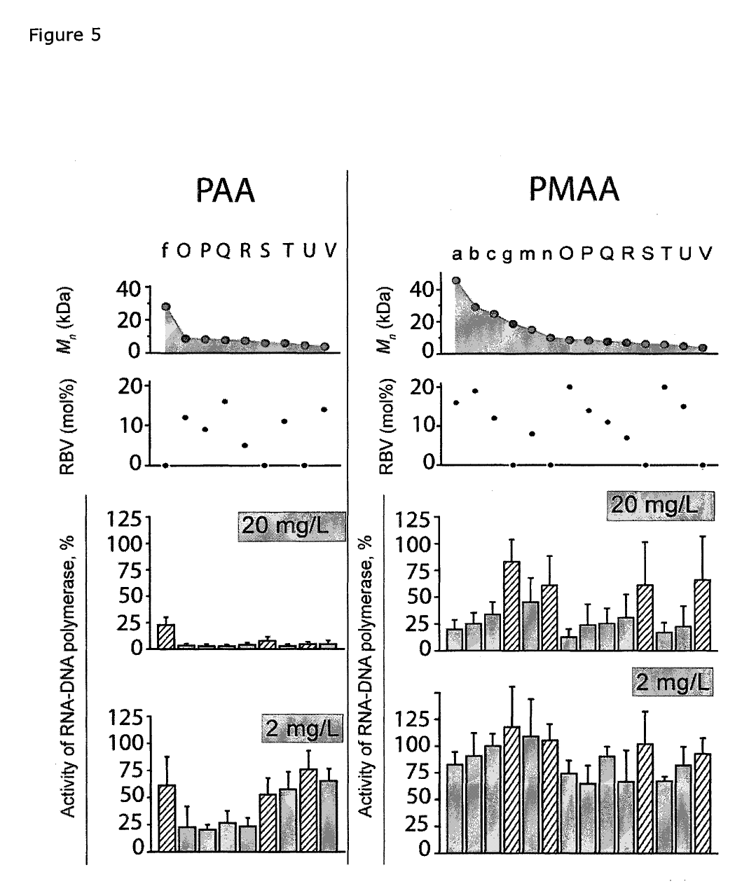

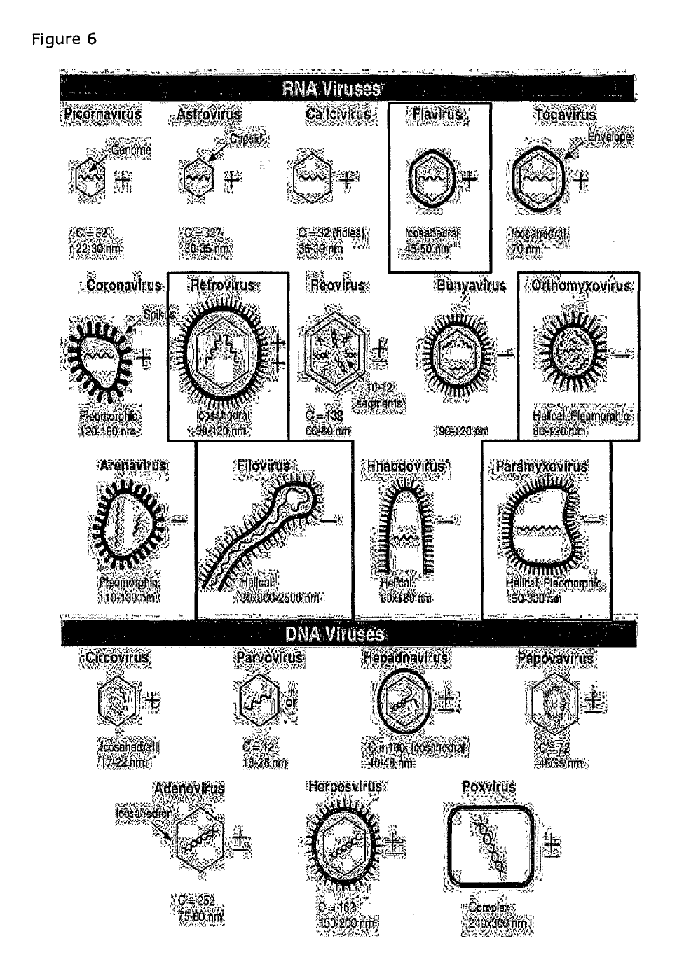

At 200 mg/L, both PAA and PMAA based polymers completely inhibited activity of the DNA polymerase and qPCR resulted in negligible quantities of de novo synthesized nucleic acid. In contrast, polymer samples with analogous structure but without anionic charge (poly-N-2(hydroxypropyl methacrylamide, HPMA and polyvinylpyrrolidone, PVP) had no activity against polymerases and qPCR remained un-inhibited (data not shown). Thus, anionic charge indeed conveys to the polymer an ability to modulate and even completely abolish activity of nucleic acid polymerase. To provide a visual proof for the above observations, standard PCR reaction was performed and results were analysed using gel electrophoresis, FIG. 4. This technique fully supported results of the qPCR and illustrates that PAA and PMAA based polymers inhibit production of DNA via a polymerase chain reaction whereas HPMA and PVP, another non-ionic polymer used in drug delivery, have no anti-polymerase activity.

Serial dilution of the polymer samples was accompanied by a gradual recovery of efficiency of qPCR and at 2 mg/L polymer content, polymers had virtually no effect on the yield of PCR (FIG. 3). At intermediate concentrations, results of qPCR present structure-function correlation for polyanions as inhibitors of polymerase activity. Among the polymers tested, four most active polymer samples were similar in that these had no ribavirin conjugated to the polymer. Indeed, in this assay, the presence of ribavirin would come at an expense of a partial loss of anionic character of the polymer (ribavirin is conjugated via an ester linkage using the PAA/PMAA carboxyl functionality). Thus, for anti-polymerase activity, anionic charge is indeed imperative. For both PAA and PMAA (pro)drug, IC50 with regards to the inhibition of PCR is ±10-20 mg/L. These values are considerably lower than polymer concentrations being active in the above antiviral assays. This observation further highlights the notion that for intracellular activity of the polymer, cell entry is a limiting step in the overall cascade of events.

The activity of the polymeric (pro)drugs was also analysed against reverse transcriptase, which is an enzyme performing the reaction of DNA synthesis from an RNA template. The same enzyme is needed for the replication of HIV and is a main target in every antiretroviral therapy in HIV-infected patients. At concentration 200 mg/L both PAA and PMAA-based (pro)drugs provided complete inhibition of reverse transcription. As in experiments with DNA polymerase, PAA (pro)drugs occurred to be more potent than PMAA based (pro)drugs. At concentration 20 mg/L the inhibition of reverse transcriptase by PAA (pro)drugs was nearly complete, but only at 2 mg/L concentration it was possible to observe structure-function relationship. The most effective PAA polymers had molecular weight in a range 9.5 to 7.3 kDa, while polymers of higher and lower molecular weight presented lower potency. Ribavirin didn't influence the activity of PAA-based (pro)drugs. Molecular weight didn't influence the activity of PMAA-based (pro)drugs. Instead, (pro)drugs with the highest content of ribavirin showed the greatest potency in inhibiting reverse transcriptase reaction. This observation was further confirmed by non-parametric Spearman's test, which showed statistically significant, negative correlation between content of ribavirin and activity of the enzyme (P=0.02).

### Methods

### Replicon Assay

PAA and PMAA libraries were screened for their antiviral activity and cellular toxicity at 0.2 g/L. It is easily observed that PAA is consistently more effective than PMAA in inhibiting viral replication. While none of the PMAA polymers show any activity at the studied concentration, the majority of (pro)drugs based on PAA show a pronounced effect on the viral genome. The activity of PAA appears to be completely independent of conjugated ribavirin, though. In all likelihood, at the investigated polymer concentration the concentration of drug delivered to the cell is not sufficient to elicit a therapeutic response. Efficacy is thus purely governed by activity of the carrier, i.e. PAA, itself. Further, this effect is strongly Mₙ dependent. A nonparametric Spearman analysis yielded a very strong correlation coefficient of 0.77 (P<0.001) between Mₙ and luminescence signal, while no correlation was observed to other factors (i.e. viability and drug loading). In fact, the overall majority of polymer samples were non-toxic to the cells and only isolated polymers had a low toxicity.

Previously, contribution of RT inhibition on the overall efficacy observed by these polymers was deemed negligible due to the poor cell penetration. It is now known that linear and dendritic polymers inhibit the replication of HIV intracellularly. In fact, this intracellular activity can conceivably proceed through the inhibition of the viral polymerase/reverse transcriptase and/or integrase. Within the replicon system employed in this study, viral replication is purely intracellular and, therefore, the mechanism of action of PAA must be intracellular. Possibly, PAA elicits its effect through a similar mechanism, a theory that is corroborated by the similarity in drug targets between HIV and HCV. This is further substantiated by the fact that dextran sulfate inhibits the activity of both viral RT as well as bacterial and cellular polymerases. Inhibition of intracellular HCV replication.

The HCV subgenomic replicon system (Con1/SG-Neo(I)hRlucFMDV2aUb, Apath, USA) in HuH7 cells (human hepatoma cell line) was maintained in full DMEM media (DMEM, 10% FBS, 1% NEA, 1% P/S, 500 μg/mL geneticin) and passaged in 2 or 3 day cycles according to the suppliers protocol through trypsinization (trypsin/EDTA 0.05%/0.02%, 5 min, 37° C.).

Effects of polymers against viral RNA replication were quantified by seeding 6000 cells/well (100 μL) of HuH7 cells harboring the replicon into a white sterile 96-well multiplate. After 2-3 h, following cellular attachment, the media was refreshed (100 μL) to remove the selection agent geneticin from the cells. Cells were incubated in the absence of G418 from here on. The agent of interest was added in triplicate on each plate to achieve the desired final concentration and the cells were incubated for further 48 hours. Subsequently, cell media was refreshed (50 μL) and a PrestoBlue viability assay conducted (Invitrogen, 10 μL, 60 min, 37° C.). 50 μL were transferred from each well to a fresh black 96-well multiplate to determine fluorescence levels (excitation 560 nm, emission 590 nm). Cell viability was calculated by normalizing blank-corrected fluorescence levels to a negative control of PBS-treated cells.

Remaining cells were washed with PBS (100 μL) and media refreshed (50 μL). 50 μL of luciferase assay reagent was added (Promega, Renilla-Glo) and luminescence was read out after 10 minutes of incubation. Luminescence was normalized to a PBS-treated cell sample.

### Inhibition of HIV-1 Replication Assay.

HIV-1 strain Bal (NIH AIDS Research and Reference Reagent Program, Bethesda, USA) was generated by transfection of HEK293T cells using calcium phosphate precipitation. Briefly, HEK293T cells were seeded at 4.5×10⁴ per cm² on T75 bottle (Nunc, Roskilde, Denmark) and 10 μg of HIV-1 plasmid mixed with 450 μL sterile water, 50 μL 2.5 M CaCl₂ and then 500 μL HEPES was added dropwise. 24 h after transfection the cell media was renewed and 48 h post transfection virus-containing supernatant was harvested, filtered through a 0.45 μm filter and stored at −80° C. TCID50 was determined by infecting TZM-bl cells and measuring luminescent signal. The calculations of TCID50 were done using Reed-Muench formula.

HeLa-derived TZM-bl cells (obtained from NIH AIDS Reagent Program, catalogue no. 8129) were used to evaluate HIV infectivity. TZM-bl cells express the HIV receptor CD4 and coreceptors CCRS and CXCR4 and harbor a luciferase β-galactosidase reporter system under the control of the HIV-1 long terminal repeats (LTRs). TZM-bl HeLa cells were maintained in Dulbecco's Modified Essential Medium (DMEM) (Lonza, Basel, Switzerland) supplemented with 10% heat-inactivated fetal calf serum (FCS), 50 U/ml penicillin and 50 μg/ml streptomycin (Invitrogen, Glostrup, Denmark). Cells were grown on T75 bottles (Nunc, Roskilde, Denmark) at 37° C. with 5% CO₂.

For the assay TZM-bl cells were seeded in 96-well flat-bottomed culture plates (Sarstedt, Newton, USA) at a density of 5000 cells per well and cultured overnight. Cells were pre-incubated with polymers at indicated concentrations for 24 h and then infected with HIV-1 strain Bal (12×TCID50 or MOI=0.1). After two days media was removed and cells were incubated with 90 μl 0.5% Nonidet P-40 (Struers Kebo Lab, Aalborg, Denmark) in PBS supplemented with 0.9 mM CaCl₂ and 0.5 mM MgCl₂ for at least 45 min in order to inactivate the virus. Luciferase activity proportional to the level of infection was measured using 90 μl of britelite plus reagent (Perkin-Elmer, Skovlunde, Denmark) per well. After mixing, 150 μl of the solution was transferred to the white 96-well plates (Perkin-Elmer, Skovlunde, Denmark). Luciferase activity was quantified by measuring luminescent signal with BritelitePlus on a FLUOstar Omega plate reader (BMG Labtech, Ortenberg, Germany).

### Inhibition of DNA-DNA Polymerase

To analyse inhibitory effect of polymers on DNA-DNA polymerase activity, quantity PCR (qPCR) reaction was performed using Power SYBR® Green PCR Master Mix (Life Technologies) according to the protocol provided by the manufacturer. For one sample 10 μl of Green PCR Master Mix were mixed with forward 5′-GGTCTCTCTGGTTAGACCAGAT-3′ and reversed primer 5′-CTGCTAGAGATTTTCCACACTG-3′, 0.46 ng of pHXB2-env plasmid (NIBSC, Programme EVA Centre for AIDS Reagents, reference number: ARP206) and a polymer diluted in PBS at a desired concentration. Primers were complementary to LTR upstream element of gag and enabled amplification of DNA fragment. The program used was 95° C. for 5 min followed by 45 cycles with 95° C. for 12 sec, 62° C. for 26 sec and during the last cycle, a melting curve was made. A level of inhibition of Taq polymerase was calculated by comparing to a positive control without polymer as 100% of enzyme activity.

The samples analysed by agarose gel electrophoresis were prepared using AmpliTaq Gold® PCR Master Mix (Applied Biosystems, Branchburg, N.J., USA). 6.25 μl of Master Mix were mixed with forward primer 5′-GGTCTCTCTGGTTAGACCAGAT-3′ and reversed primer 5′-CTGCTAGAGATTTTCCACACTG-3′, 46 ng of pHXB2-env plasmid (NIBSC, Programme EVA Centre for AIDS Reagents, reference number: ARP206) and a polymer solution at the final concentration 200 mg/L. The program used was 95° C. for 5 min followed by 30 cycles with 94° C. for 15 sec, 50° C. for 15 sec, 72° C. for 30 sec, and last cycle 72° C. for 7 min. Samples were separated on 2% agarose gel and signal from nucleic acids was visualised with Gel Red Nucleic Acid Stain (Biotium).

### Inhibition of RNA-DNA Polymerase

Influence of polymers on activity of reverse transcriptase was determined using EnzChek® Reverse Transcriptase Assay Kit (Life Technologies). The reaction mixture was prepared according to the protocol provided by the manufacturer. Subsequently polymers diluted in PBS were added at the given concentration, and then 5 U of MuLV Reverse Transcriptase (Life Technologies, Cat. Nr. N8080018). The reaction was performed at 37° C. for 1 hr. Nucleic acids were stained with PicoGreen dye and fluorescence was measured on a plate reader (BMG Labtech, Ortenberg, Germany).

### Example 2

Polyanionic macromolecular prodrug of ribavirin has a broad spectrum of activity covering HIV, Ebola, Influenza, respiratory syncytial virus, measles, and mumps.

TABLE
Polymers used in this work

| | | | RBV | |
|---|---|---|---|---|
| No | Carrier | Mₙ (kDa) | (mol %) | characteristics |
| 1 | PAA | 21 | 11 | RBV conjugated via a labile linkage |
| 2 | PAA | 31 | 0 | |
| 3 | PAA | 13 | 0 | |
| 4 | PAA | 14 | 18 | RBV conjugated via a labile linkage |
| 5 | PAA | 6 | 0 | |
| 6 | PAA | 8 | 16 | RBV conjugated via a labile linkage |
| 7 | PMAA | 25 | 17 | RBV conjugated via a labile linkage |
| 8 | PMAA | 17 | 16 | RBV conjugated via a labile linkage |
| 9 | PMAA | 19 | 0 | |
| 10 | PMAA | 6 | 0 | |
| 11 | PMAA | 8 | 10 | RBV conjugated via a labile linkage |
| 12 | PMAA | 14 | 4 (SIL) | ultra-sensitive linkage |
| 13 | PMAA | 28 | 5 (SIL) | ultra-sensitive linkage |
| 14 | PSS | 70 | 0 | Sulphonated polymer control |

The ultra-sensitive linker (used in polymers 12 and 13) corresponds to a linker having a self-immolative spacer and a disulfide bond.

### Summary of the Virus Silencing Assays

Polymers are added 1-2 h before the virus; virus is left to incubate with cells for 48 h (measles and mumps) or 72 h (WSN, ebola, RSV, dengue). Good effect seen on Influenza (Orthomyxoviridae), Measles (Paramyxoviridae), Mumps and Ebola; NOT Dengue (Flavivirus). All of these viruses have a viral envelope consisting of host cell membrane (negative charge) with specific viral proteins embedded which are positively charged. MPCL also has data on inhibition against HIV and HCV.

| | | | | | Polymer |
|---|---|---|---|---|---|
| | | | | | inhibition |
| | | | | | at |
| | | | | | 200 mg/L |
| | | | MOI tested | | Low virus |
| Virus | Genus | Family | Low | High | dose |
| Influenza | Influenza A | Orthomyxoviridae | 0.0001 | 0.001 | 12, 13 |
| Dengue | Serotype 2 | Flaviviradae | | N/A | 6 |
| Measles | Morbillivirus | Paramyxoviridae | 1 | 2 | 12, 13 |
| Mumps | Rubulavirus | Paramyxoviridae | 0.5 | 1 | 13 |
| Ebola | Zaire | Filoviridae | 0.5 | N/A | 1, 2, 4, 7, |
| | | | | | 12, 13 |
| RSV | Pneumovirus | Paramyxoviridae | 0.001 | N/A | All |
| | | | | | except 1 |
| HCV | Hepacivirus | Flaviviradae | | | |
| HIV | Lentivirus | Retroviridae | | | |

Influenza

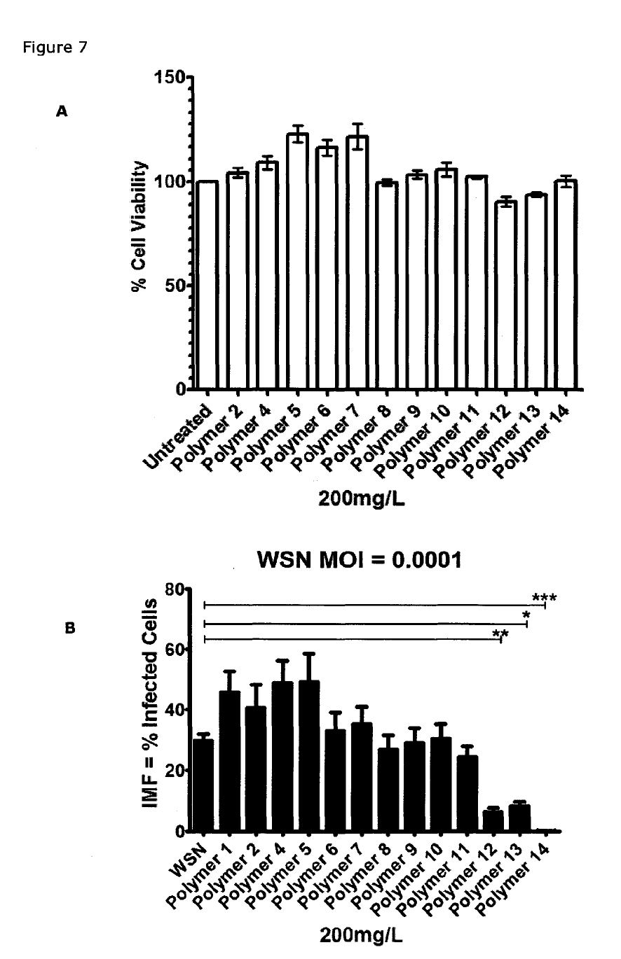

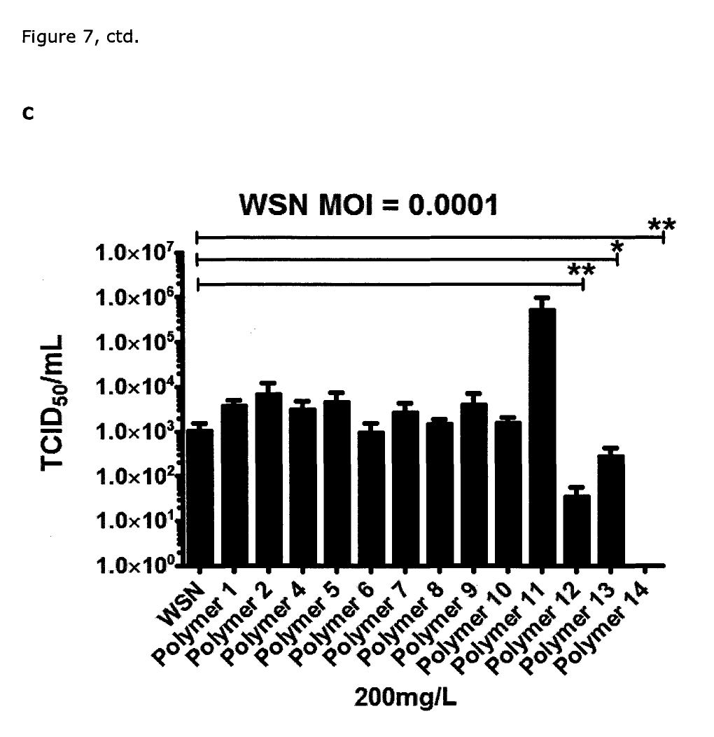

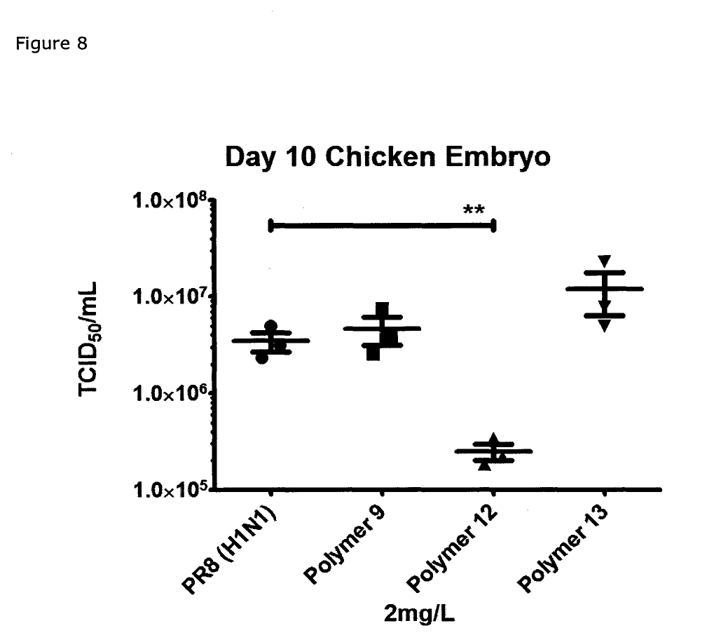

Cf. FIGS. 7 and 8.

Experiments are run in A549 lung cancer cells.

Quantification of the virus infection in terms of number of cells testing positive for the proliferating virus was performed using a Cell Insight visualization platform and intracellular immunohistochemical labelling of the virus (expressed as IMF values).

TCID50 endpoint dilution assay quantifies the amount of virus required to kill 50% of infected hosts. Thus, it is a virus quantification assay.

In these experiments, PSS seems to be the most effective polymer. At the same time, PAA and PMA polymers are not active and in fact upon incubation with these polymers, an increase in the number of cells positive for the internalized virus is observed. This is also reflected by an increase in TCID50 values, i.e. an increase in the amount of produced viral particles.

In contrast, the cells treated with ribavirin-SIL polymers revealed a statistically significant ˜10-fold decrease in the TCID50 and the fraction of cells with the virus detected in the cells. Corresponding parent PMA with no ribavirin on the chain or with ribavirin connected via ester linkages had no effect—illustrating the superior efficacy of polyanionic macromolecular prodrug (PAMP) based on disulfide trigger for drug release.

Respiratory Syncytial Virus (RSV)

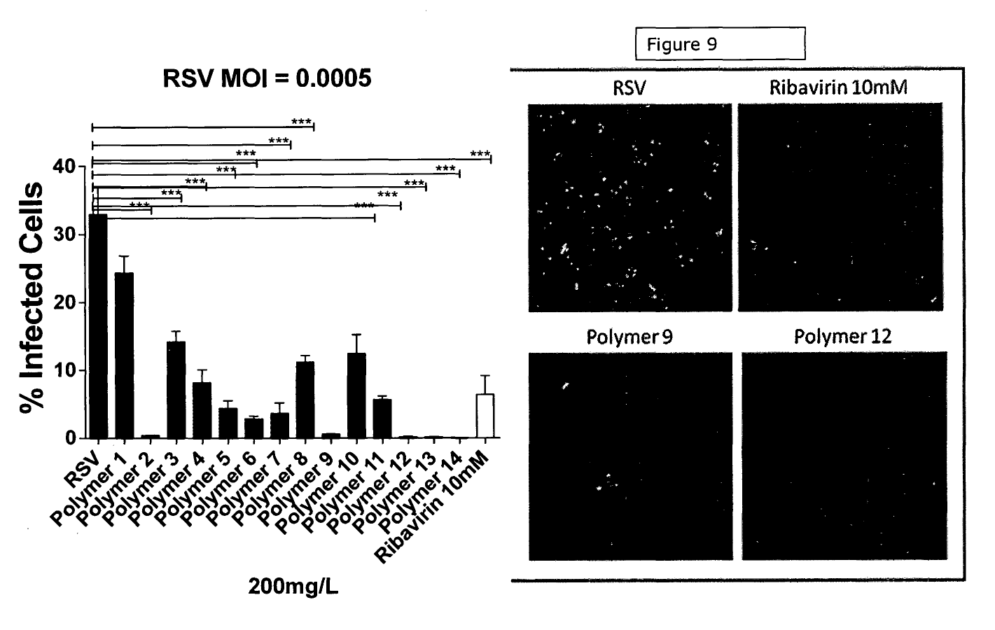

Cf. FIG. 9

Measles

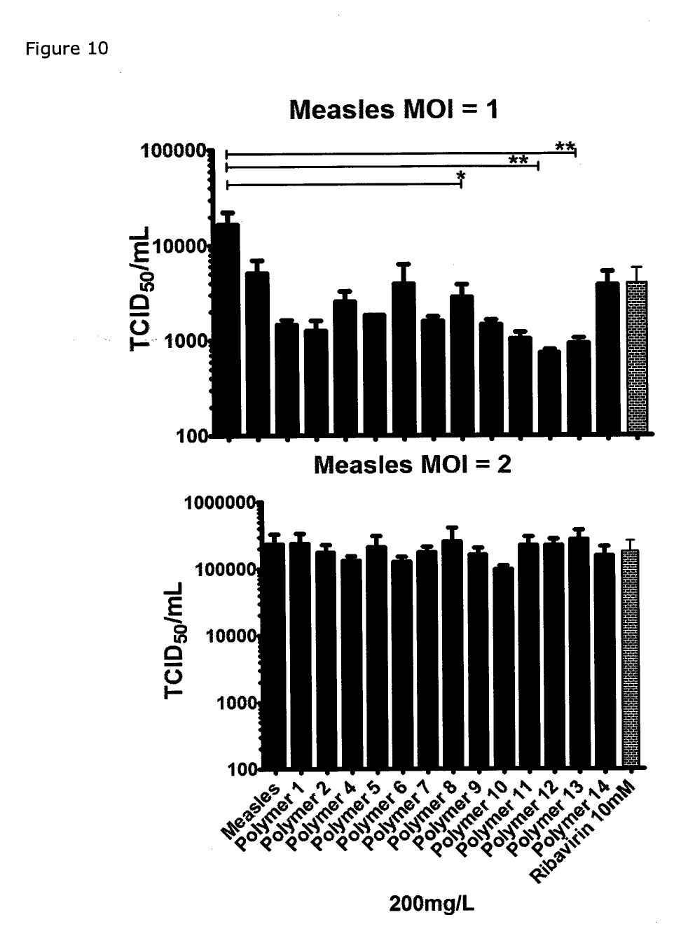

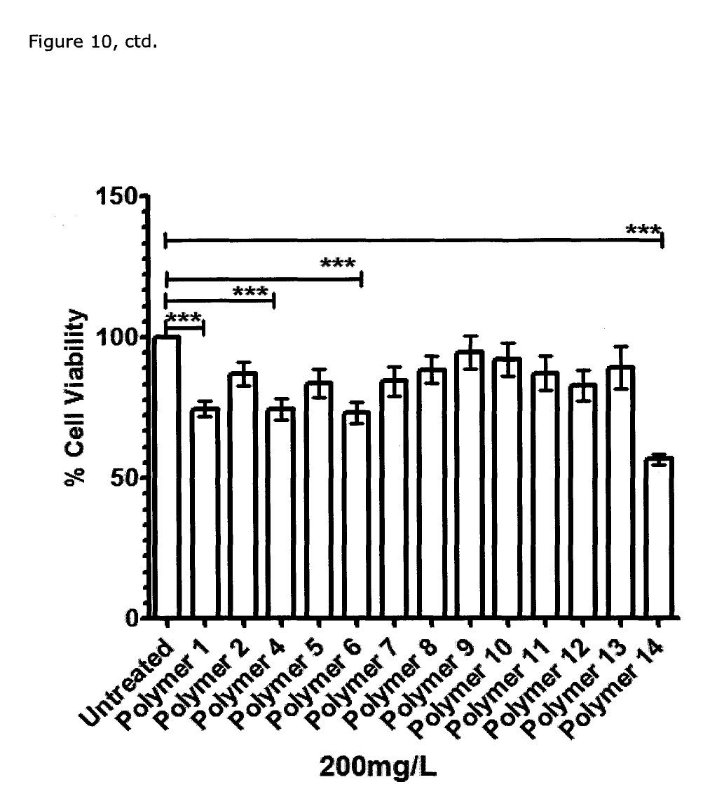

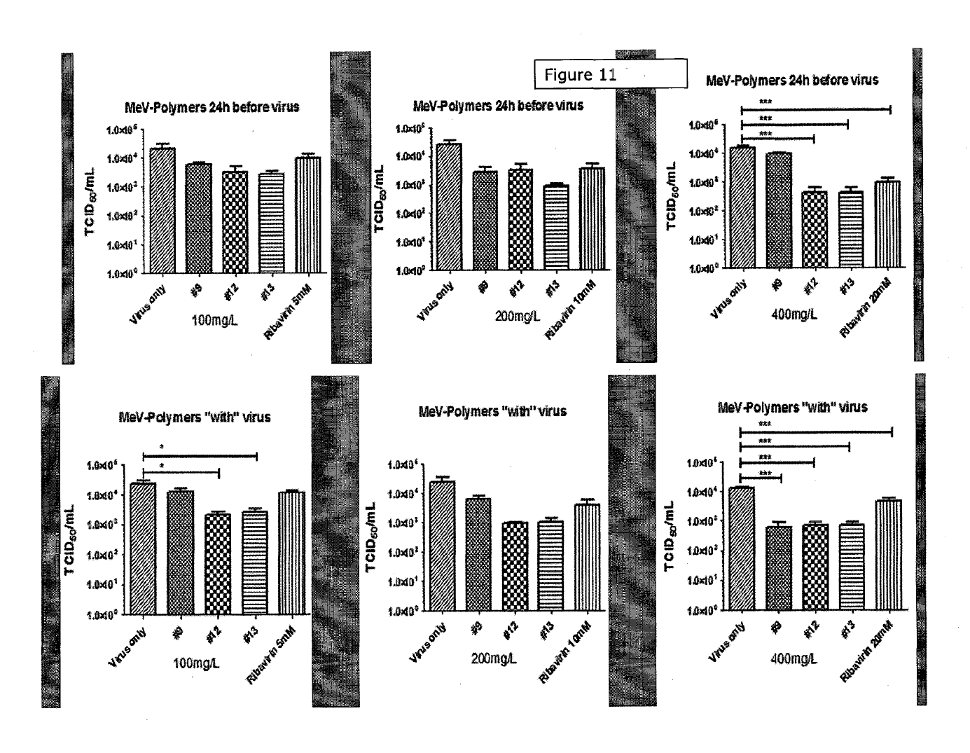

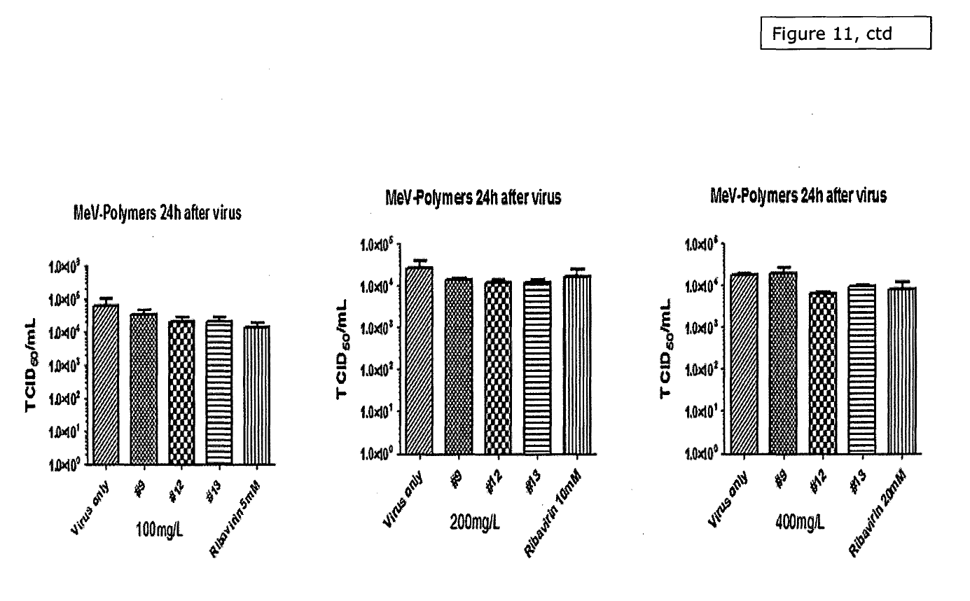

Cf. FIG. 10 and FIG. 11.

Ribavirin itself (not conjugated) showed no activity; PSS (no drug) showed no activity—illustrating that purely electrostatic mode of activity—i.e. prevention of viral cell entry—is not sufficient to prevent infectivity of measles. Of the acrylic PAMP, polymers with no ribavirin were most effective and the presence of the drug was detrimental to the inhibitory effect. The same does not hold true for methacrylic PAMP and polymers 12 and 13 based on the disulfide trigger and self-immolative linkage (SIL) were most effective. Surprisingly, increasing MOI to 2 (2 viral particles per mammalian cell taken for initial virus inoculation) nullified the polymer effects.

Toxicity of the polymers with and without the drug was quantified in HeLa cells used as hosts for measles and mumps infection. Toxicity was most pronounced for PSS and was not significant for polymers with the highest antiviral activity, polymers 12 and 13.

Detailed characterization of antiviral effects against measles was designed to address the three possible relative timepoints of polymer administration compared to the administration of the virus: polymer addded prior to virus inoculation (preventative measures); virus and the polymer added at the same time; polymer added 24 h after virus inoculation (therapeutic treatment). This was done for 3 polymer concentrations (100, 200, and 400 ug/mL). The latter of the treatments had no effect and TCID50 values were not decreased by the administered polymers. Pre-treatment with polymers resulted in a pronounced inhibition of measles infectivity (as much as 10-fold decrease in TCID50). The strongest effect was observed upon co-administration of the polymer and the virus—suggesting that prevention of virus cell entry is a major component in the treatment of measles with the proposed PAMP. However, the data obtained for pre-treatment with the polymers taken at a high concentration (400 ug/mL) also reveal that PAMP were 10-100-fold more effective than the parent, drug-free polymer (PMAA free of ribavirin) thus revealing that ribavirin is also playing a considerable part in the overall therapeutic activity of PAMP.

Mumps

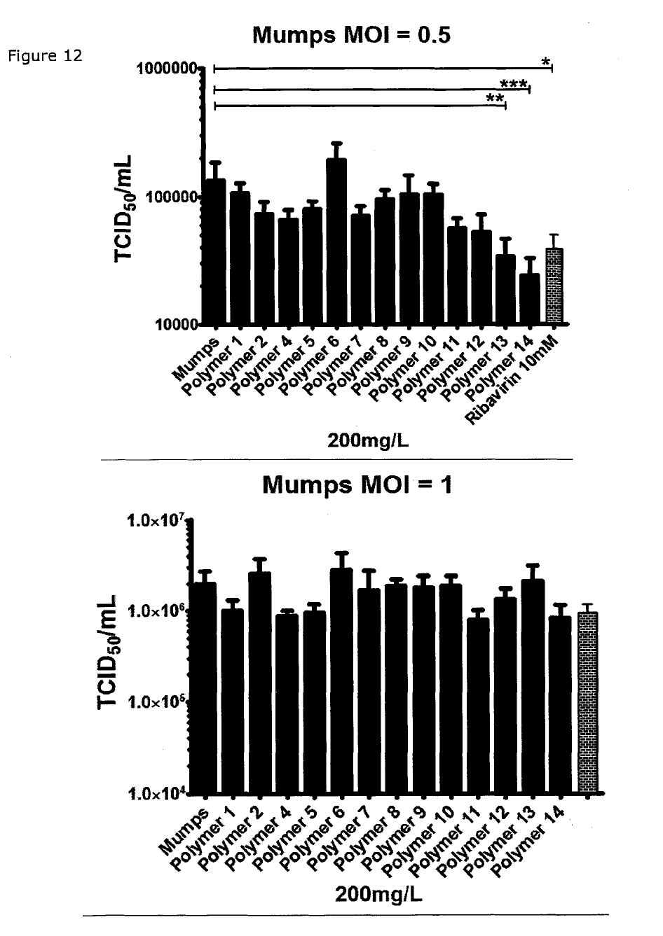

Cf. FIG. 12

For mumps, a similar pattern emerged to that discussed above for the inhibition of infectivity of measles. PAMP containing ribavirin on the disulfide, SIL equipped linker were markedly, statistically significantly more effective in preventing infectivity of this virus than the parent polymer or PAMP with ribavirin linked via an ester functionality.

Ebola

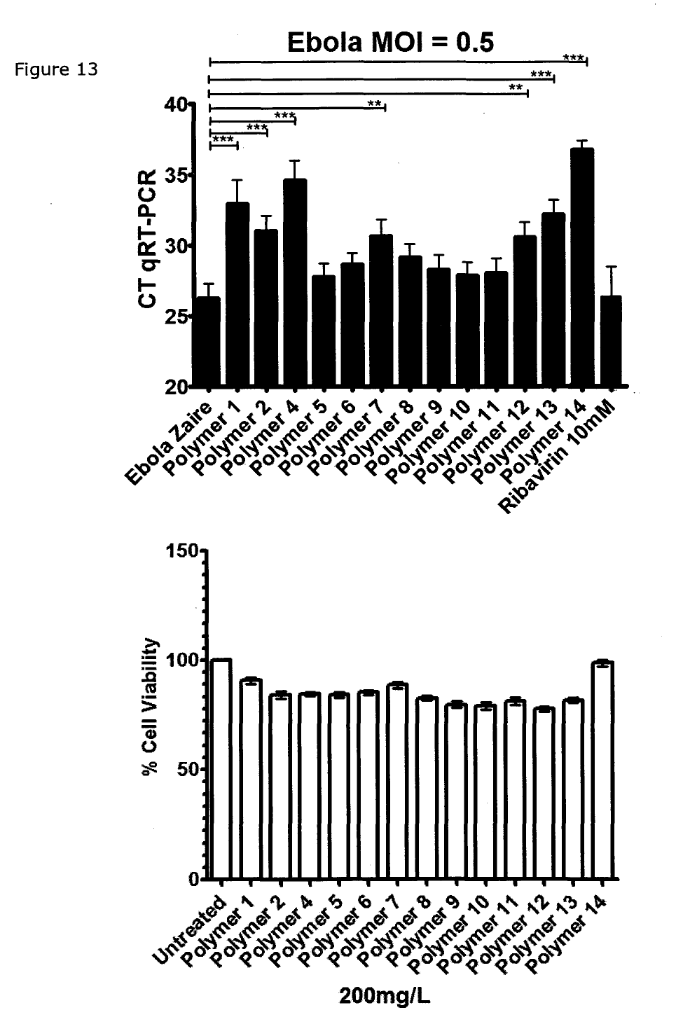

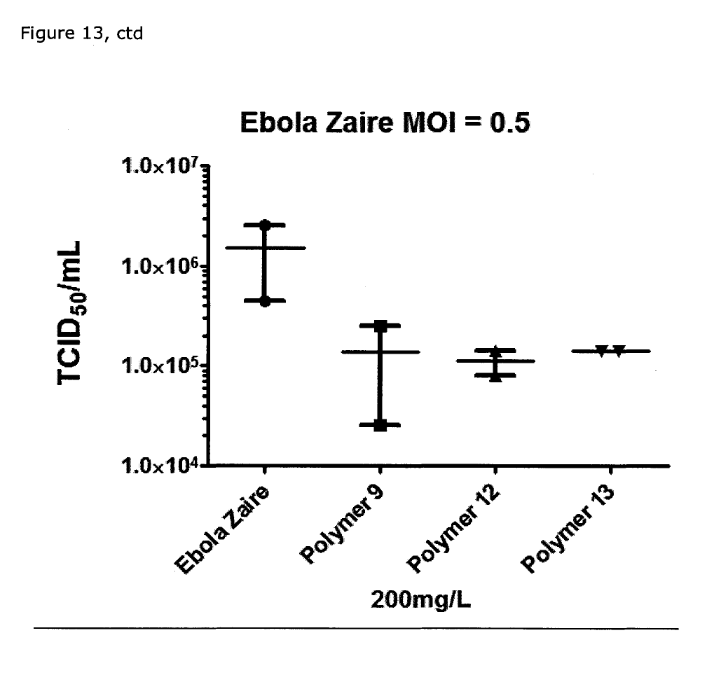

Cf. FIG. 13

For Ebola virus, virus proliferation was quantified in VERO cells and using qRT-PCR as a readout. In this assay, an increase in the cycle number at which amplification goes over the threshold of detection implies positive therapeutic effect, i.e. decrease in the proliferation of the virus. Ribavirin itself had no measurable effect on the proliferation of Ebola virus. PSS, a strong polyanion, had the strongest effect revealing that electrostatic component is a major, possibly by far the strongest contributor to the overall antiviral effect. Interestingly, of the PAMP, the strongest virus inhibition was observed not for the pristine polymers but for the ribavirin containing conjugates. Of these, methacrylic PAMP containing ribavirin on the disulfide, SIL linker proved to be stat significantly effective in preventing infectivity and proliferation of Ebola. Viral load was also quantified using an end point analysis (TCID50) and this approach too revealed that polymers provided an over 10-fold decrease in the amount of the synthesized viral particles.

### Example 3

Synthesis of the disulfid SIL-RBV monomer and its copolymerization with methacrylic acid

### Monomer Synthesis:

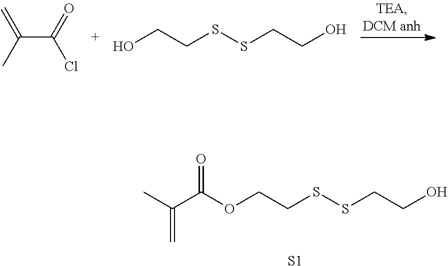

TEA (0.57 mL, 4.09 mL) has been added dropwise over a cold solution of 2-hydroxyethyl disulfide (0.50 mL, 4.09 mmol) in DCM (12 mL) under N2 atmosphere. Then, methacryloyl chloride (0.20 mL, 2.05 mmol) has been added dropwise. The reaction has been stirred for 1 h warming it slowly to rt. It has been followed by TLC (pentane:EtOAc 7:3, with a visualization solution of KMnO4). The reaction has been washed with NH4Cl sat. and brine, and dried over MgSO4 anh. The crude has been purified with a silica column (pentane:EtOAc, from 90:10 to 70:30) affording S1 (0.34 g, 74%). 1H-NMR (400 MHz, DMSO-d6): δ (ppm) 6.05 (s, 1H), 5.70 (s, 1H), 4.87 (t, J=5.4 Hz, 1H), 4.34 (t, J=6.3 Hz, 2H), 3.62 (dt, J=5.4 and 6.4 Hz, 2H), 3.01 (t, J=6.3 Hz, 2H), 2.81 (t, J=6.4 Hz, 2H), 1.88 (s, 3H). HRMS (ESI+) calculated for C8H15O3S2 m/z (M+H+) 223.0457, found 223.0456; C8H14O3S2Na m/z (M+Na+) 245.0277, found 245.0276.

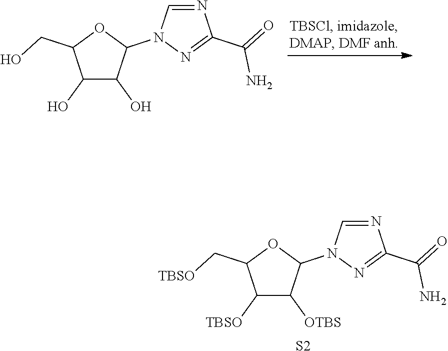

Imidazole (0.40 g, 5.93 mmol) and DMAP (26 mg, 0.21 mmol) have been added over a solution of RBV (0.21 g, 0.85 mmol) in DMF anh. (2.0 mL) under N2 atmosphere. The solution has been stirred for 5 minutes. A solution of TBSCI (0.77 g, 5.09 mmol) in DMF (1.4 mL) has been added over the previous solution. The reaction has been stirred for 24 h at rt, it has been followed by TLC (DCM:EtOAc 50:50, with a visualization solution of KMnO4). Then, the reaction has been diluted with DCM and washed with NH4Cl sat. (3 times), and brine (once), and dried over Na2SO4 anh. The crude has been purified through a column (pentane:EtOAc from 80:20 to 60:40) affording the compound S2 (0.42 g, 84% yield). 1H-NMR (400 MHz, DMSO-d6): δ (ppm) 8.90 (s, 1H), 7.67 (d, J=30.7 Hz, 2H), 5.84 (d, J=5.5 Hz, 1H), 4.65 (dd, J=4.6 and 5.5 Hz, 1H), 4.28-4.34 (m, 1H), 3.95-4.01 (m, 1H), 3.67- 3.82 (m, 2H), 0.91 (s, 9H), 0.86 (s, 9H), 0.77 (s, 9H), 0.12 (s, 3H), 0.10 (s, 3H), 0.10 (s, 3H), 0.05 (s, 3H), 0.04 (s, 3H), −0.03 (s, 3H), −0.21 (s, 3H). 13C-NMR (100 MHz, DMSO-d6): δ (ppm) 160.1 (s), 157.5 (s), 145.9 (d), 90.3 (d), 85.7 (d), 75.1 (d), 72.0 (d), 62.4 (t), 25.8 (3q), 25.7 (3q), 25.5 (3q), 18.0 (s), 17.7 (s), 17.6 (s), −4.7 (q), −4.8 (q), −4.9 (q), −5.5 (3q). HRMS (ESI+) calculated for C26H55N4O5Si3 m/z (M+H+) 587.3475, found 587.3478; C26H54N4O5Si3Na m/z (M+Na+) 609.3294, found 609.3298.

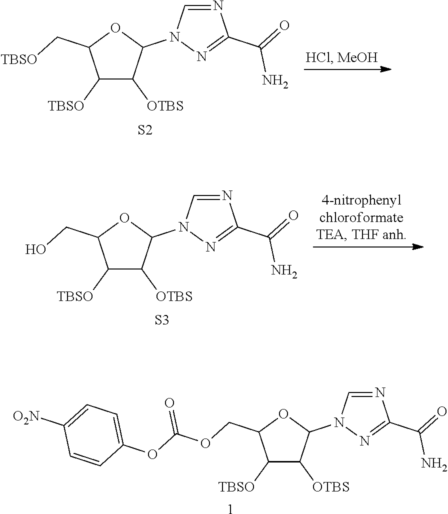

A solution of 1.25 M HCl in MeOH (7.1 mL, 8.91 mmol) has been added over a solution of S2 (4.36 g, 7.43 mmol) in MeOH (74 mL). The reaction has been stirred for 2 hours at rt. A white compound has precipitated and has been filtered and washed with cold MeOH. The collected fraction of MeOH has been concentrated affording more amount of the white compound, which has been filtered and washed with cold MeOH. Both precipitates have been collected affording the compound S3 (2.55 g, 73%). TEA (0.24 mL, 1.74 mmol) has been added over a suspension of S3 (0.64 g, 0.87 mmol) in THF (4 mL). 4-nitrophenyl chloroformate (0.26 g, 1.30 mmol) has been added over the previous suspension after 5 min under N2 atmosphere. The reaction has been stirred at rt for 20 hours. The solvent has been removed under vacuum and the crude has been dissolved in EtOAc. The organic solution has been washed with brine and dried with MgSO4 anh. The crude has been purified with a silica column (pentane:EtOAc from 70:30 to 60:40) affording 1 (0.47 g, 85%). 1H-NMR (400 MHz, CDCl3): δ (ppm) 8.40 (s, 1H), 8.28 (d, J=9.1 Hz, 2H), 7.37 (d, J=9.1 Hz, 2H), 7.00 (s, 1H), 6.17 (s, 1H), 5.80 (d, J=2.6 Hz, 1H), 4.63 (dd, J=3.8, 13.2 Hz, 1H), 4.55-4.59 (m, 1H), 4.40 (dd, J=3.8, 13.2 Hz, 1H), 4.32-4.38 (m, 2H), 0.89 (s, 9H), 0.92 (s, 9H), 0.10 (s, 6H), 0.08 (s, 3H), 0.02 (s, 3H). 13C-NMR (100 MHz, CDCl3): δ (ppm) 160.8 (s), 157.5 (s), 155.3 (s), 152.4 (s), 145.7 (s), 144.4 (d), 125.5 (2d), 121.9 (2d), 92.9 (d), 81.7 (d), 76.2 (d), 71.2 (d), 67.1 (t), 25.9 (3q), 25.8 (3q), 18.1 (2s), −4.2 (q), −4.5 (q), −4.7 (q), −4.9 (q). HRMS (ESI+) calculated for C27H44N5O9Si2 m/z (M+H+) 638.2672, found 638.2672; C27H43N5O9Si2Na m/z (M+Na+) 660.2492, found 660.2493.

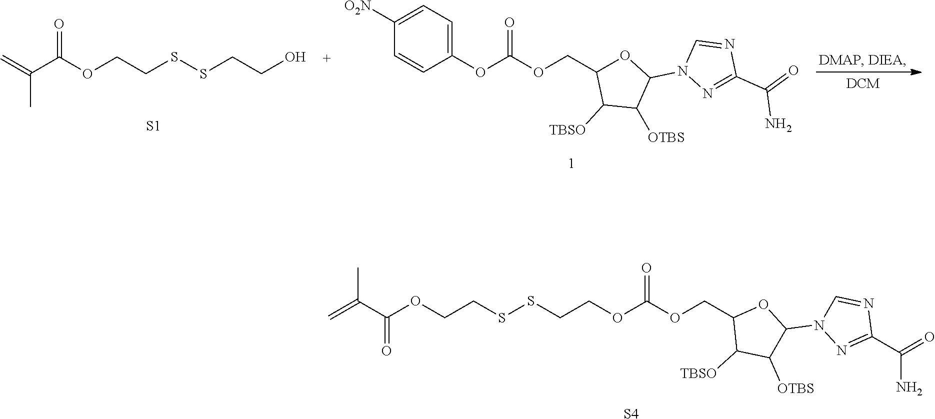 [SMILES: C=C(C)C(=O)OCCSSCCO ; "S1" ; CC(C)(C)[Si](C)(C)OC1C(COC(=O)Oc2ccc([N+](=O)[O-])cc2)OC(n2cnc(C(N)=O)n2)C1O[Si](C)(C)C(C)(C)C ; C=C(C)C(=O)OCCSSCCOC(=O)OCC1OC(n2cnc(C(N)=O)n2)C(O[Si](C)(C)C(C)(C)C)C1O[Si](C)(C)C(C)(C)C ; "S4"]

A solution of 1 (0.66, 1.03 mmol) in DCM (20 mL) has been added over a colorless solution of S1 (0.46 g, 2.06 mmol), DIEA (0.54 mL, 3.09 mmol), and DMAP (0.03 g, 0.21 mmol) in DCM (60 mL) under N2 atmosphere. Then, the reaction mixture has turned into yellow. The reaction has been stirred at rt for 26 hours, and it has been followed by NMR. The crude has been washed with NH4Cl sat. (2×) and brine (2×), and dried over MgSO4 anh. The crude has been purified with a silica column (pentane:EtOAc, from 80:20 to 50:50) affording the compound S4 (0.78 g, quantitative). This compound is stable for a week inside the fridge, but a small addition of hydroquinone (as inhibitor) is suggested if the compound is stored for long time. 1H-NMR (400 MHz, CDCl3): δ (ppm) 8.40 (s, 1H), 7.01 (s, 1H), 6.15-6.11 (m, 1H), 5.78 (d, J=2.9 Hz, 1H), 5.72 (bs, 1H), 5.61-5.57 (m, 1H), 4.54-4.47 (m, 2H), 4.42 (td, J=2.5 and 6.6 Hz, 4H), 4.33-4.23 (m, 3H), 2.97 (dt, J=6.6 and 11.1 Hz, 4H), 1.95 (s, 3H), 0.91 (s, 9H), 0.88 (s, 9H), 0.08 (s, 3H), 0.07 (s, 6H), 0.02 (s, 3H). 13C-NMR (100 MHz, CDCl3): δ (ppm) 167.3 (s), 160.6 (s), 157.3 (s), 154.7 (s), 144.2 (d), 136.1 (s), 126.2 (t), 93.0 (d), 82.0 (d), 76.4 (d), 71.1 (d), 66.2 (t), 66.0 (t), 62.7 (t), 37.4 (t), 37.0 (t), 25.9 (3q), 25.8 (3q), 18.4 (q), 18.1 (2s), 14.34, −4.2 (q), −4.5 (q), −4.7 (q), −4.9 (q). HRMS (ESI+) calculated for C29H53N4O9S2Si2 m/z (M+H+) 721.2787, found 721.2793; C29H52N4O9S2Si2Na m/z (M+Na+)743.2606, found 743.2643.

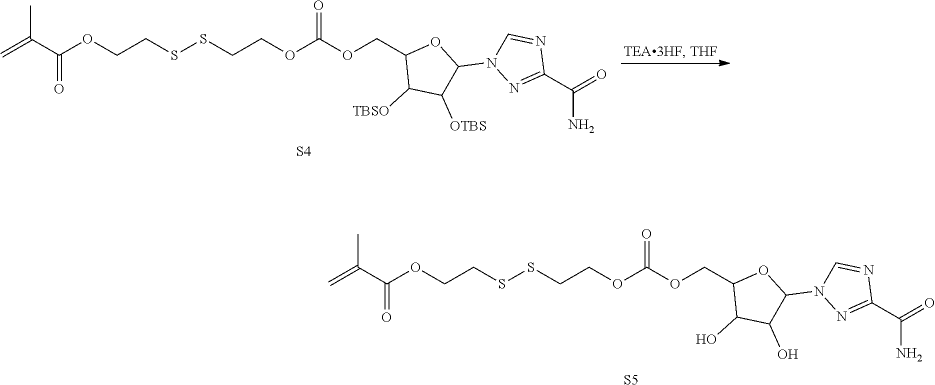 [SMILES: C=C(C)C(=O)OCCSSCCOC(=O)OCC1OC(n2cnc(C(N)=O)n2)C(O[Si](C)(C)C(C)(C)C)C1O[Si](C)(C)C(C)(C)C ; "S4" ; C=C(C)C(=O)OCCSSCCOC(=O)OCC1OC(n2cnc(C(N)=O)n2)C(O)C1O ; "S5"]

TEA·3HF (0.18 mL, 1.44 mmol) has been added over a solution of S4 (0.16 g, 0.29 mmol) in THF anh. (3.2 mL) under N2 atmosphere. The reaction has been stirred at rt for 24 h, and followed by NMR. The solvent has directly been removed under vacuum, then, the crude has been diluted with DCM and purified twice with two silica column (firstly, DCM:MeOH, from 100:1 to 100:2, secondly, DCM:MeOH 100:2). S5 has been afforded with an 84% yield (0.09 g). 1H-NMR (400 MHz, CDCl3): δ (ppm) 8.81 (s, 1H), 7.84 (s, 1H), 7.64 (s, 1H), 6.04 (s, 1H), 5.90 (d, J=2.8 Hz, 1H), 5.69 (s, 1H), 4.44-4.24 (m, 7H), 4.23-4.07 (m, 2H), 3.34 (bs, 2H), 3.06-2.93 (m, 4H), 1.87 (s, 3H). 13C-NMR (100 MHz, CDCl3): δ (ppm) 166.4 (s), 160.3 (s), 157.6 (s), 154.2 (s), 145.4 (d), 135.6 (s), 126.1 (t), 91.5 (d), 81.5 (d), 74.1 (d), 70.4 (d), 68.0 (t), 65.4 (t), 62.2 (t), 36.3 (t), 36.1 (t), 17.9 (q). HRMS (ESI+) calculated for C17H25N4O9S2m/z (M+H+) 493.1057, found 493.1106; C17H24N4O9S2Na m/z (M+Na+) 515.0877, found 515.0913.

Polymer Synthesis:

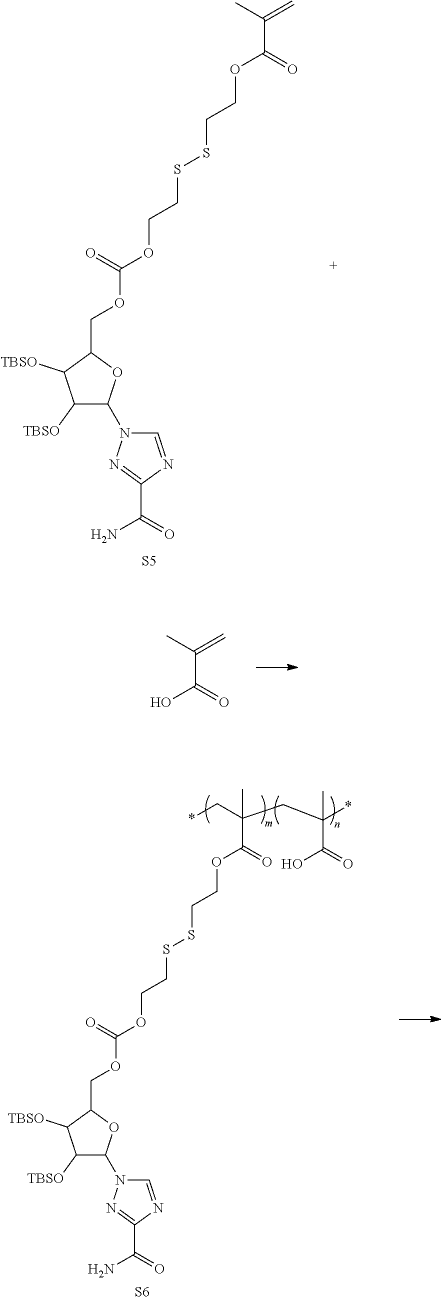

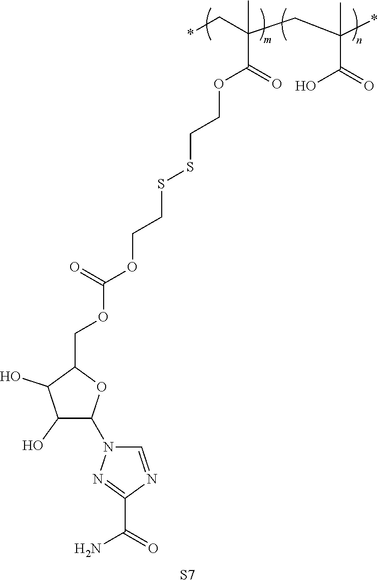

S5 and freshly distilled methacrylic acid have been dissolved in DMF and mixed inside a glass ampule. Stock solutions of AlBN (21.4 mg/mL) and 2-cyano-2-propyl benzodithioate (109.1 mg/mL) have been prepared and added to that mixture (See Table 2 for amounts). A small amount of 1,3,5-trioxane has been added to the mixture as an internal standard. The ampule has been then degassed via three freeze-pump-thaw cycles and flame-sealed under vacuum. The ampule has been heated for 6 hours at 60oC to start the polymerization. The reaction has been quenched opening the ampule. The crude has been precipitated into DCM:MeOH (95:5), affording a thin pink powder (S6), it has been filtered, washed with more DCM and dried with N2. TEA·3HF (0.15 mL, 0.92 mmol) has been added over a solution of S6 (50 mg) in DMF (0.40 mL). The reaction has been stirred at rt for 12 h and it has been followed by NMR. The polymer has been precipitated with a mixture DCM:MeOH (95:5) affording a pinky precipitate S7 (0.028 g).

TABLE 2

| | | | | | | 2-cyano- | |
|---|---|---|---|---|---|---|---|
| | | | | | | 2-propyl | |
| | MAA | | S5 | | AIBN | benzodithioate | DMF |
| | V | | m | | V | V | V |
| | (mL) | mmol | (mg) | mmol | (mL) | (mL) | (mL) |
| PMAA-2 | 0.146 | 1.72 | 72 | 0.091 | 0.051 | 0.054 | 0.188 |
| PMAA-3 | 0.156 | 1.84 | 77 | 0.097 | 0.027 | 0.029 | 0.257 |
| PMAA-4 | 0.142 | 1.67 | 70 | 0.088 | 0.012 | 0.013 | 0.259 |

1-27. (canceled)

28. A compound comprising a polyanion carrier conjugated to an antiviral drug via a biodegradable linker, wherein the compound has antiviral activity by at least two modes of antiviral activities selected from: i) extracellular inhibition of virus cell entry due to the activity of polyanion carrier; ii) intracellular inhibition of the viral polymerases due to activity of the polyanion carrier; and iii) intracellular antiviral activity due to release of the antiviral drug from the carrier upon cell entry.

29. The compound according to claim 1, wherein the biodegradable linker comprises a disulfide bond and a self-immolative spacer.

30. The compound according to claim 1, wherein the linker is capable of releasing the antiviral drug via disulfide reshuffling in the presence of a thiol.

31. The compound according to claim 1, wherein the antiviral drug is a broad-spectrum antiviral drug.

32. The compound according to claim 1, wherein the antiviral drug is a nucleoside or ribonucleoside analogue.

33. The compound according to claim 1, wherein said antiviral drug is a ribavirin, azdothymidine, lamivudine, favipiravir or a derivative thereof of any of the foregoing.

34. The compound according to claim 1, wherein the polyanion carrier comprises monomers selected from the group consisting of: poly(acrylic acid) and poly(methacrylic) acid.

35. The compound according to claim 1, for use in the treatment, amelioration or prevention of a retroviral infection.

36. The compound according to claim 1, for use in the treatment, amelioration or prevention of one or more of influenza, HIV, hepatitis C virus, ebola, mumps, respiratory syncytial virus, dengue and measles.

37. A compound comprising a polyanion carrier conjugated to an antiviral drug via a biodegradable linker, wherein the compound has antiviral activity, wherein the antiviral activity is a attributed to the combined effects of the effects of the antiviral activity of the polyanion carrier and the antiviral activity of the antiviral drug.

38. The compound according to claim 37, wherein the biodegradable linker comprises a disulfide bond and a self-immolative spacer.

39. The compound according to claim 37, wherein the linker is capable of releasing the antiviral drug via disulfide reshuffling in the presence of a thiol.

40. The compound according to claim 37, wherein the antiviral drug is a broad-spectrum antiviral drug.

41. The compound according to claim 37, wherein the antiviral drug is a nucleoside or ribonucleoside analogue.

42. The compound according to claim 37, wherein the antiviral drug is a ribavirin, azdothymidine, lamivudine, favipiravir or a derivative thereof of any of the foregoing.

43. The compound according to claim 37, wherein the polyanion carrier comprises monomers selected from the group consisting of: poly(acrylic acid) and poly(methacrylic) acid.

44. The compound according to claim 37, for use in the treatment, amelioration or prevention of a retroviral infection.

45. The compound according to claim 37, for use in the treatment, amelioration or prevention of one or more of influenza, HIV, hepatitis C virus, ebola, mumps, respiratory syncytial virus, dengue and measles.


SEQUENCE LISTING (2 sequences)

SEQ ID NO: 1 - DNA, 22 bp, Artificial Sequence
  Primer
ggtctctctggttagaccagat

SEQ ID NO: 2 - DNA, 22 bp, Artificial Sequence
  primer
ctgctagagattttccacactg